(19) United States
(12) Patent Application Publication
(10) Pub. No.: US 2011/0160488 A1
(43) Pub. Date: Jun. 30, 2011

(54) Fluorination Processes with Arylsulfur Halotetrafluorides

(75) Inventors: Teruo Umemoto, Westminster, CO (US); Rajendra P. Singh, Broomfield, CO (US)

(73) Assignee: I M &T RESEARCH, INC., Denver, CO (US)

(21) Appl. No.: 12/525,714

(22) PCT Filed: Mar. 6, 2009
(86) PCT No.: PCT/US09/36305

§ 371 Date: Aug. 4, 2009

Related U.S. Application Data
(60) Provisional application No. 61/041,415, filed on Apr. 1, 2008.
(60) Provisional application No. 61/034,600, filed on Mar. 7, 2008.

(51) Int. Cl. C07C 319/20

(52) U.S. Cl. 568/74

(57) ABSTRACT

New fluorination processes for introducing one or more fluorine atoms into target substrate compounds with arylsulfur halotetrafluorides are disclosed. Also disclosed are methods for preparation of arylsulfur trifluorides.

### TECHNICAL FIELD

The present invention relates to new fluorination processes using arylsulfur halotetrafluoride as a fluorinating agent.

### BACKGROUND OF THE INVENTION

Fluorine-containing compounds have found wide use in medical, agricultural, electronic and other like industries (see Chemical & Engineering News, June 5, pp 15-32 (2006); Angew. Chem. Ind. Ed., Vol. 39, pp 4216-4235 (2000)). These compounds show specific biologic activity or physical properties based on the presence of one or more fluorine atoms. A particular drawback in their usefulness is the scarcity of natural fluorine-containing compounds, requiring most such compounds to be prepared through organic synthesis.

Fluorinating agents are compounds that selectively introduce fluorine atom(s) into target compounds through one or more chemical reactions to produce fluorine-containing compounds. Particularly useful fluorinating agents have the capacity to replace oxygen or oxygen-containing groups or sulfur or sulfur-containing groups in the target compound with fluorine. A number of fluorinating agents have been discovered; however, as discussed in more detail below, these agents generally have significant drawbacks based on safety, reactivity, stability, production, handling, storage and/or disposability.

Illustrative examples of known fluorinating agents include: sulfur tetrafluoride (SF₄), a highly toxic gas that is often utilized under pressure [J. Am. Chem. Soc., Vol. 82, pp 543-551 (1960)]; N,N-diethylaminosulfur trifluoride (DAST), an unstable liquid agent having a highly explosive nature, i.e., low thermal stability and large amounts of thermal energy upon decomposition [J. Org. Chem., Vol. 40, pp 574-578 (1975) and Chem. & Eng. News, Vol. 57, No. 19, p 4 (1979)]; bis(methoxyethyl)aminosulfur trifluoride (Deoxo-Fluor®) or its N-aryl analogs, compounds that have low thermal stability [U.S. Pat. No. 6,222,064 B1; Chem. Commun., pp 215-216 (1999); J. Org. Chem. Vol. 65, pp 4830-4832 (2000)]; selenium tetrafluoride (SeF₄), a highly toxic selenium compound [J. Am. Chem. Soc., Vol. 96, pp 925-927 (1974)]; and various other designed fluorinating agents that provide greater safety but have provided substantially reduced reactivity and yields: e.g., phenylfluorophosphane reagents [PhₙPF₅₋ₙ (n=1˜3), Chem. Pharm. Bull., Vol. 16, p 1009 (1968)], α,α-difluoroalkylamino reagents [ClCFHCF₂NEt₂, Organic Reactions, Vol. 21, pp 158-173 (1974); CF₃CFHCF₂NEt₂, Bull. Chem. Soc. Jpn, Vol. 52, pp 3377-3380 (1979); CF₂HCF₂NMe₂, J. Fluorine Chem., Vol. 109, pp 25-31 (2001)], 2,2-difluoro-1,3-dimethylimidazolidine [Chem. Commun., pp 1618-1619 (2002)], and [(m-methylphenyl)difluoromethyl]diethylamine (Tetrahedron, Vol. 60, pp 6923-6930); phenylsulfur trifluoride was used as a fluorinating agent, but its fluorination yields have proven low and its applicability narrow [J. Am. Chem. Soc., Vol. 84, pp 3058-3063 (1962); Acta Chimica Sinica, Vol. 39, No. 1, pp 63-68 (1981)]. Pentafluorophenylsulfur trifluoride was used only for conversion of benzaldehyde to (difluoromethyl)benzene [J. Fluorine Chem., Vol. 2, pp 53-62 (1972/73)] and, recently, multi-substituted phenylsulfur trifluorides were reported as fluorinating agents [U.S. Pat. No. 7,265,247 B1]. Requirement for pentafluorophenyl and multi-substituted phenyl parts increases the cost. In addition to the above-mentioned drawbacks, most of the fluorinating agents mentioned above are extremely or substantially sensitive to moisture. Therefore, production and handling of these materials is extremely troublesome since moisture or wet conditions easily decompose the fluorinating agents. Recently, it appeared that multi-substituted phenylsulfur trifluorides such as 4-tert-butyl-2,6-dimethylphenylsulfur trifluoride in solid state are not sensitive to water (see, for example, U.S. Pat. No. 7,381,846 B2). However, 4-tert-butyl-2,6-dimethylphenylsulfur trifluoride in solution is very sensitive to water; immediate decomposition occurs on contact with water.

Each of these conventional, illustrative fluorinating agents, or production methods, allows for room for improvement on providing easy, safe, effective, and less costly fluorination agents or methods for the production of these important fluorine-containing compounds.

As such, there is a need in the field to provide, safe, reactive, less hazardous, easily produced/stored, cost effective, fluorinating reagents or methods, especially fluorinating agents and methods that selectively introduce fluorine atoms into compounds in high yields and that are insensitive to moisture.

The present invention is directed toward overcoming one or more of the problems discussed above.

### SUMMARY OF THE INVENTION

The present invention provides new fluorination processes with arylsulfur halotetrafluorides as fluorinating agents for the introduction of fluorine atoms into target compounds. The resultant target compounds, i.e., fluorine-containing compounds, have shown tremendous potential in the medical, agricultural, electronic and other like industries.

In general, arylsulfur halotetrafluoride compounds are used as fluorinating agents. Typical arylsulfur halotetrafluoride compounds are substituted or unsubstituted phenylsulfur halotetrafluorides. Among these compounds, substituted or unsubstituted phenylsulfur chlorotetrafluorides are preferred. The substituted or unsubstituted phenylsulfur halotetrafluorides are shown herein to have substantial functional, safety, and ease of handling advantages over compounds and methods utilizing conventional fluorinating agents.

In particular, compounds of the present invention have enhanced stability due to capacity to avoid degradation due to moisture or wet conditions.

The present invention also provides new and useful preparative processes for unsubstituted or substituted phenylsulfur trifluorides.

These and various other features as well as advantages which characterize the invention will be apparent from a reading of the following detailed description and a review of the appended claims.

### DETAILED DESCRIPTION OF THE INVENTION

The present invention provides new processes for introducing one or more fluorine atoms into target compounds with arylsulfur halotetrafluorides as fluorinating agents. In the present invention the term “target compound” includes any substrate that once fluorinated is useful in the medical, agricultural, biological, electronic materials' or other like field, i.e., is a fluorine-containing compound. In preferred instances, the target compound(s) of the invention include one or more oxygen atom(s) and/or one or more oxygen-containing group(s), and/or one or more sulfur atom(s) and/or one or more sulfur-containing group(s) for selective replacement by the fluorine atom(s). The target compound(s) of the invention also include other functional groups or moieties for substitution or addition by one or more fluorine atoms. In some cases, the fluorination may occur with other halogenation such as chlorination. Preferred illustrative target compounds include alcohols, silyl ethers, aldehydes, ketones, carboxylic acids, acid halides, esters, acid anhydrides, amides, imides, epoxides, lactones, lactams, sulfonic acids, sulfinic acids, sulfinyl halides, sulfenic acids, sulfenyl halides, thiols, sulfides, sulfoxides, thioketones, thioesters, dithioesters, thiocarboxylic acids, thiocarbonyl halides, dithiocarboxylic acids, thiocarbonates, dithiocarbonates, trithiocarbonates, thioketals, dithioketals, thioacetals, dithioacetals, thioamides, thiocarbamates, dithiocarbamates, orthothioesters, phosphines, phosphine oxides, phosphine sulfides, phosphonic acids, and other like compounds, and salts thereof.

Typical fluorination processes herein include: a one-step process, a one-step process with reducing agent, and a two-step process with a reducing substance.

In general, the invention provides processes for introducing one or more fluorine atoms into a target compound with arylsulfur halotetrafluoride represented by the formula (I):

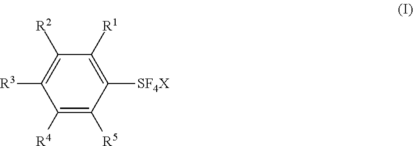

wherein X is a chlorine atom, bromine atom, or iodine atom, and R¹, R², R³, R⁴, and R⁵ each is independently a hydrogen atom, a halogen atom, a substituted or unsubstituted alkyl group having one to ten carbon atoms, a nitro group, a cyano group, a substituted or unsubstituted aryl group having six to sixteen carbon atoms, a substituted or unsubstituted alkanesulfonyl group having one to ten carbon atoms, a substituted or unsubstituted arenesulfonyl having six to sixteen carbon atoms, a substituted or unsubstituted alkoxy group having one to ten carbon atoms, a substituted or unsubstituted aryloxy group having six to sixteen carbon atoms, or a SF₅ group.

Arylsulfur halotetrafluoride compounds of formula (I) include isomers such as trans-isomers and cis-isomers as shown below; arylsulfur halotetrafluoride is represented by ArSF₄X:

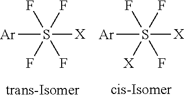

Note that according to the nomenclature of Chemical Abstract Index Name, for example, C₆H₅—SF₄Cl is named sulfur, chlorotetrafluorophenyl-; p-CH₃—C₆H₄—SF₄Cl is named sulfur, chlorotetrafluoro(4-methylphenyl)-; and p-NO₂—C₆H₄—SF₄Cl is named sulfur, chlorotetrafluoro(4-nitrophenyl)-.

Arylsulfur halotetrafluoride compounds as represented by the formula (I) are stable and insensitive to moisture, and thus safe and easy to handle. Phenylsulfur chlorotetrafluoride maintains high stability on contact with water. When a solution of phenylsulfur chlorotetrafluoride in chloroform-d is contacted with water at room temperature, about 74% of phenylsulfur chlorotetrafluoride remains after 3 hours, and the half life time of phenylsulfur chlorotetrafluoride in chloroform-d in contact with water is estimated to be approximately 8 hours. Conventional fluorinating agents such as DAST, Deoxo-Fluor, and phenylsulfur trifluoride decompose immediately and violently with sound emission and fume production when placed in contact with water. When a solution of useful fluorinating agent, 4-tert-butyl-2,6-dimethylphenylsulfur trifluoride (see, for example, U.S. Pat. No. 7,381,846 B2) in a solvent (chloroform-d) is contacted with water, immediate decomposition occurs. Therefore, the arylsulfur halotetrafluorides of the invention is easily isolated during production and can easily be used as a fluorinating agent. In one embodiment, the arylsulfur halofluorides are prepared at low cost by treating the corresponding diaryl disulfides or arylthiols with chlorine (Cl₂) in the presence of a metal fluoride (see Examples 1˜12).

With reference to Formula (I) again (including the trans and cis-isomers): Preferably, X is a chlorine atom from a viewpoint of cost.

A halogen atom of R¹, R², R³, R⁴, or R⁵ refers to a fluorine, chlorine, bromine, or iodine atom. Among them, fluorine, chlorine, and bromine atoms are preferable, and chlorine is most preferable from a viewpoint of cost.

When used herein, the term “alkyl” includes all straight, branched, and cyclic isomers. Representative examples of alkyl groups having one to ten carbon atoms include methyl, ethyl, propyl, isopropyl, cyclopropyl, butyl, isobutyl, sec-butyl, tert-butyl, pentyl, isopentyl, neopentyl, tert-pentyl, cyclopentyl, hexyl, cyclohexyl, heptyl, octyl, nonyl, decyl, and so on. More preferred alkyl groups have one to four carbon atoms, and methyl, ethyl, propyl, isopropyl, butyl, isobutyl, sec-butyl, tert-butyl are exemplified. Among them, methyl and tert-butyl are furthermore preferred.

Preferred substituted alkyl groups include; fluorinated and/or chlorinated alkyl groups such as CF₃, CCl₃, CF₂H, CFH₂, CClH₂, and CF₃CF₂, and alkoxy-substituted alkyl groups such as CH₃OCH₂, CH₃CH₂OCH₂, CH₃CH₂CH₂OCH₂, (CH₃)₂CH₂OCH₂, CH₃(CH₂)₂CH₂OCH₂, (CH₃)₂CH₂CH₂OCH₂, CH₃CH(CH₃)CH₂OCH₂, CH₃OCH₂CH₂, and CH₃CH₂OCH₂CH₂.

Substituted and unsubstituted aryl groups herein have six to sixteen carbon atoms. The term “aryl” includes phenyl and naphthyl, with preferred aryl groups being phenyl. Preferred substituted aryl groups include; alkylated, fluorinated, chlorinated, brominated, nitrated, and/or trifluoromethylated phenyl groups such as methylphenyl, ethylphenyl, propylphenyl, butylphenyl, dimethylphenyl, fluorophenyl, chlorophenyl, nitrophenyl, (trifluoromethyl)phenyl, and so on.

Substituted and unsubstituted alkanesulfonyl groups have one to ten carbon atoms. The term “alkanesulfonyl” is a binding of “alkyl” described above and a sulfonyl (SO₂) group, that is, alkylsulfonyl. As “alkyl” in alkanesulfonyl (alkylsulfonyl) group, these are exemplified the same as alkyl above. Preferred examples of alkanesulfonyl groups include CH₃SO₂, CH₃(CH₂)ₙSO₂ (n=1˜3), (CH₃)₂CHSO₂, CH₃CH(CH₃)CH₂SO₂, and (CH₃)₂CHCH₂SO₂. Preferred examples of substituted alkanesulfonyl groups include fluorinated alkanesulfonyl groups such as CF₃SO₂.

Substituted and unsubstituted arenesulfonyl groups herein have six to sixteen carbon atoms. The term “arenesulfonyl” is a binding of “aryl” described above and a sulfonyl (SO₂) group, that is, arylsulfonyl. For “aryl” in arenesulfonyl (arylsulfonyl) group, these are exemplified the same as aryl above. Arylsulfonyl groups include phenylsulfonyl and naphthylsulfonyl, with preferred arylsulfonyl groups being phenylsulfonyl. Preferred examples of substituted arenesulfonyl groups include alkylated, fluorinated, chlorinated, brominated, nitrated, and/or trifluoromethylated phenylsulfonyl groups, e.g. methylphenylsulfonyl, dimethylphenylsulfonyl, fluorophenylsulfonyl, chlorophenylsulfonyl, bromophenylsulfonyl, and nitrophenylsulfonyl.

Substituted and unsubstituted alkoxyl groups herein have one to ten carbon atoms. The term “alkoxy” is a binding of “alkyl” described above and an oxygen atom, that is, alkyloxyl. Representative examples of alkoxy (alkyloxy) groups having one to ten carbon atoms include CH₃O, CH₃(CH₂)ₙO (n=1˜9), (CH₃)₂CHO, (CH₃)₂CHCH₂O, CH₃CH₂(CH₃)CHO, (CH₃)₃CO, CH₃CH₃(CH₃)CHCH₂O, and (CH₃)₃CCH₂O. More preferred alkoxyl groups have one to four carbon atoms, and CH₃O, CH₃CH₂O, CH₃CH₂CH₂O, (CH₃)₂CHO, CH₃CH₂CH₂CH₂O, CH₃CH₂(CH₃)CHO, (CH₃)CHCH₂O, and (CH₃)₃CO are exemplified.

Preferred substituted alkoxyl groups include; fluorinated and/or chlorinated alkoxyl groups such as CF₃O, CF₃CH₂O, CCl₃CH₂O, CF₃CF₂O, CF₂HCF₂O, and (CF₃)₂CHO, and alkoxy-substituted alkoxy groups such as CH₃OCH₂CH₂O.

Substituted and unsubstituted aryloxy groups herein have six to sixteen carbon atoms. The term “aryloxy” is a binding of “aryl” described above and an oxygen atom. As “aryl” in aryloxy group, which is exemplified the same as aryl above. Aryloxy groups include phenyloxy and naphthyloxy, with preferred aryloxyl groups being phenyloxyl. Preferred examples of substituted aryloxy groups include alkylated, fluorinated, chlorinated, brominated, nitrated, and/or trifluoromethylated phenyloxy groups such as methylphenyloxy, fluorophenyloxy, chlorophenyloxy, bromophenyloxy, nitrophenyloxy, and (trifluoromethyl)phenyloxy.

Preferably, from a cost perspective, arylsulfur halotetrafluoride is selected from a group consisting of arylsulfur halotetrafluorides in which R¹, R², R³, R⁴, and R⁵ each is independently selected from a group consisting of a hydrogen atom, a halogen atom, a substituted or unsubstituted linear or branched alkyl group having one to four carbon atoms, and a nitro group. More preferably, arylsulfur halotetrafluoride is selected from a group consisting of arylsulfur chlorotetrafluorides in which R¹, R², R³, R⁴, and R⁵ each is independently selected from a group consisting of a hydrogen atom, a halogen atom, a substituted or unsubstituted linear or branched alkyl group having one to four carbon atoms, and a nitro group. Furthermore preferably, arylsulfur halotetrafluoride is selected form a group consisting of arylsulfur halotetrafluorides in which R¹, R², R³, R⁴, and R⁵ are all a hydrogen atom and at maximum three of R¹, R², R³, R⁴, and R⁵ each is independently selected from a group consisting of a halogen atom, a substituted or unsubstituted linear or branched alkyl group having one to four carbon atoms, and a nitro group, and the remainders of R¹, R², R³, R⁴, and R⁵ are a hydrogen atom. Additionally preferably, arylsulfur halotetrafluoride is selected form a group consisting of arylsulfur chlorotetrafluorides in which R¹, R², R³, R⁴, and R⁵ are all a hydrogen atom and at maximum three of R¹, R², R³, R⁴, and R⁵ each is independently selected from a group consisting of a halogen atom, a substituted or unsubstituted linear or branched alkyl group having one to four carbon atoms, and a nitro group, and the remainders of R¹, R², R³, R⁴, and R⁵ are a hydrogen atom.

Furthermore preferably embodiments herein include, arylsulfur halotetrafluoride selected from a group consisting of phenylsulfur chlorotetrafluoride, o-, m-, and p-alkylphenylsulfur chlorotetrafluoride wherein the alkyl is a linear or branched alkyl group having one to four carbon atoms, o-, m-, and p-fluorophenylsulfur chlorotetrafluorides, o-, m-, and p-chlorophenylsulfur chlorotetrafluorides, o-, m-, and p-bromophenylsulfur chlorotetrafluorides, o-, m-, and p-nitrophenylsulfur chlorotetrafluoride, and each isomer of difluorophenylsulfur chlorotetrafluoride. Isomers of difluorophenylsulfur chlorotetrafluorides include 2,3-, 2,4-, 2,5-2,6-, 3,4-, and 3,5-difluorophenylsulfur chlorotetrafluorides. Among these, phenylsulfur chlorotetrafluoride, p-methylphenylsulfur chlorotetrafluoride, p-(tert-butyl)phenylsulfur chlorotetrafluoride, p-fluorophenylsulfur chlorotetrafluoride, p-chlorophenylsulfur chlorotetrafluoride, p-bromophenylsulfur chlorotetrafluoride, and p-nitrophenylsulfur chlorotetrafluoride are more preferable, and phenylsulfur chlorotetrafluoride, p-methylphenylsulfur chlorotetrafluoride, p-(tert-butyl)phenylsulfur trifluoride, p-chlorophenylsulfur chlorotetrafluoride, and p-nitrophenylsulfur chlorotetrafluoride are furthermore preferable, and additionally, phenylsulfur chlorotetrafluoride is most preferable because of cost.

In one embodiment, a one-step process for introducing one or more fluorine atoms into a target compound is provided comprising contacting a target compound with arylsulfur halotetrafluoride represented by the formula (I). With respect to compounds represented by formula (I):

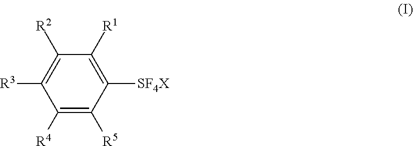

X, R¹, R², R³, R⁴, and R⁵ are the same as described and exemplified above.

This method is a one-step fluorination of a target compound with an arylsulfur halotetrafluoride.

The target compounds are described as above. Among them, preferable target compounds for this one-step fluorination are sulfur-containing compounds such as thioketones, thioesters, dithioesters, thiocarboxylic acids, thiocarbonyl halides, dithiocarboxylic acids, thiocarbonates, dithiocarbonates, trithiocarbonates, thioketals, dithioketals, thioacetals, dithioacetals, thioamides, thiocarbamates, dithiocarbamates, orthothioesters, sulfenyl halides, thiols, sulfides, and other like compounds.

The arylsulfur halotetrafluorides are described above.

In some cases, the one-step fluorination is conducted in the presence of an acid such as a Brönsted acid or a Lewis acid in order to accelerate reaction or to increase fluorination yields. The amount of acid used in any given reaction depends on the target compound(s), other chemical compounds and/or reaction conditions. Preferably, the Brönsted acid is at least one member selected from a group consisting of hydrogen fluoride (HF), HBF₄, HBCl₄, HBFCl₃, HSbF₄, HSbFCl₃, HSbF₆, HSbFCl₅, HSbF₄Cl₂, HN(SO₂CF₃)₂, and other like acids, and their complexes with organic compounds such as ethers, amines, and so on, and mixtures thereof. As complexes of HF with amines, there are preferably exemplified a mixture of HF and pyridine, a mixture of HF and α, β, and/or γ-methylpyridine, a mixture of HF and dimethylpyridine, a mixture of HF and trimethylpyridine, a mixture of HF and trimethylamine, a mixture of HF and triethylamine, and so on. Among complexes with amines, an about 70:30 wt % mixture of HF and pyridine, a 3:1 molar ratio mixture of HF and triethylamine, and a 5:1 molar ratio mixture of HF and triethylamine are more preferable because of availability. Preferably, Lewis acids used herein include a member selected from a group consisting of BF₃, BCl₃, SbF₃, SbCl₃, SbF₆, SbCl₆, SbF₃Cl₂, SnCl₄, SnF₄, SnCl₃F, TiF₄, TiCl₄, and other like acids, and their complexes with organic compounds such as ethers, nitriles, and so on, as well as mixtures thereof. As the preferred complexes, BF₃ etherates are exemplified.

In some cases, the one-step fluorination is conducted in the presence of a base in order to increase production yields, or when starting materials and/or products are sensitive to acid conditions. Preferable bases are exemplified with metal fluorides such as sodium fluoride, potassium fluoride, cesium fluoride, and other like compounds; carbonates such as sodium carbonate, sodium bicarbonate, potassium carbonate, potassium bicarbonate, and other like compounds. The amount of base used in any given reaction depends on the target compound(s), other chemical compounds and/or reaction conditions.

The amount used of arylsulfur halotetrafluoride is dependent on the kind and nature of the target compound and reaction conditions such as temperature, solvent, and catalyst or additive used. Therefore, one can choose the amount necessary for obtaining an adequate yield of the fluorinated compound from the target compound in each reaction. An illustrative amount used of arylsulfur halotetrafluoride per one mole of a target compound is from about 0.5 moles to about ten moles and more typically about one mole to about five moles.

Fluorination is conducted in the presence or absence of a solvent. In some cases, reaction can preferably be conducted without a solvent. In other cases, solvent may be used for mild or selective fluorination, and solvent is preferably selected from the group consisting of hydrocarbons, halocarbons, ethers, nitriles, aromatics, nitro compounds, esters, and mixtures thereof. Example hydrocarbons include normal, branched, cyclic isomers of pentane, hexane, heptane, octane, nonane, decane, dodecane, undecane, and other like compounds. Illustrative halocarbons include; dichloromethane, chloroform, carbon tetrachloride, dichloroethane, trichloroethane, terachloroethane, trichlorotrifluoroethane, chlorobenzene, dichlorobenzene, trichlorobenzene, hexafluorobenzene, benzotrifluoride, and bis(trifluoromethyl)benzene; normal, branched, cyclic isomers of perfluoropentane, perfluorohexane, perfluoroheptane, perfluorooctane, perfluorononane, and perfluorodecane; perfluorodecalin; and other like compounds. Illustrative ethers include diethyl ether, dipropyl ether, di(isopropyl)ether, dibutyl ether, t-butyl methyl ether, tetrahydrofuran, dioxane, glyme (1,2-dimethoxyethane), diglyme, triglyme, and other like compounds. Illustrative nitriles include acetonitrile, propionitrile, benzonitrile, and other like compounds. Illustrative aromatics include benzene, toluene, xylene, and other like compounds. Illustrative nitro compounds include nitromethane, nitroethane, nitrobenzene, and other like compounds. Illustrative esters include methyl acetate, methyl propionate, ethyl acetate, ethyl propionate, propyl acetate, isopropyl acetate, butyl acetate, isobutyl acetate, sec-butyl acetate, tert-butyl acetate, and other like compounds.

Preferably, reaction temperature for fluorination can be selected in the range of about −80° C. to about +200° C., and more preferably, about −50° C. to about +150° C. The reaction temperature depends on the use of arylsulfur halotetrafluorides, target compounds, solvents, catalysts and/or additives. Therefore, the temperature is determined from the reaction conditions necessary for the reaction.

The reaction time is also dependent on reaction temperature, target compounds, arylsulfur halotetrafluorides, solvents, catalysts or additives, and their amounts used. Therefore, one can choose the time necessary for completing each reaction by modifying one or more these parameters, but is generally from about 1 minute to several days, and is preferably within a few days.

In another embodiment, a one-step process with reducing agent is provided for introducing one or more fluorine atoms into a target compound, comprising contacting a target compound with arylsulfur halotetrafluoride represented by the formula (I) in the presence of a reducing substance.

This method is a one-step fluorination of a target compound with an arylsulfur halotetrafluoride in the presence of a reducing substance.

The target compounds and arylsulfur halotetrafluorides are described as above.

A reducing substance in accordance with this embodiment is an element or an organic or inorganic compound which reduces arylsulfur halotetrafluoride of the formula (I) used in the reaction or of which reduction potential is lower than that of arylsulfur halotetrafluoride of the formula (I) used in the reaction. One or more reducing compounds can be used in a reaction.

Reducing substances herein include elements such as: metals such as alkali metals (elements in Group 1 of the Periodic Table), alkali earth metals (elements in Group 2 of the Periodic Table), transition metals and inner transition metals (elements in Groups 3˜12 of the Periodic Table), and metals in Groups 13˜15 of the Periodic Table such as Al, Ga, In, Tl, Sn, Pb, and Bi; semi-metals such as B, Si, Ge, As, Sb, Te, Po, and At; nonmetal elements in Groups 13˜17 of the Periodical Table (C, P, S, Se, I, and so on). Among these, preferred elements are alkali metals, alkali earth metals, transition metals, metals in Groups 13˜15 of the Periodic Table, semi-metals, and nonmetals.

Reducing substances herein also include inorganic compounds such as; hydrogen, metal compounds, semi-metal compounds, and nonmetal compounds. Among these, preferred inorganic compounds include; metal salts; semi-metal salts; nonmetal salts; inorganic chloride salts; inorganic bromide salts; inorganic iodide salts; ammonia (NH₃); inorganic sulfur compounds; and so on.

Preferred inorganic chloride salts are exemplified with metal chlorides (LiCl, NaCl, KCl, RbCl, CsCl, MgCl₂, MgClF, CaCl₂, TiCl₂, VCl₂, CrCl₂, FeCl₂, CuCl, SnCl₂, and other metal salts containing chloride anions), ammonium chloride, and other inorganic salts containing chloride anions. Preferred inorganic bromide salts are exemplified with metal bromides (LiBr, NaBr, KBr, RbBr, CsBr, MgBr₂, MgBrCl, MgBrF, CaBr₂, FeBr₂, CuBr, SnBr₂, and other metal salts containing bromide anions), ammonium bromide, and other inorganic salts containing bromide anions. Preferred inorganic iodide salts are exemplified with metal iodides (LiI, NaI, KI, RbL, CsI, MgI₂, MgBrI, MgClI, MgFI, CaI₂, FeI₂, CuI, SnI₂, and other metal salts containing iodide anions), ammonium iodide, and other inorganic salts containing iodide anions. Preferred inorganic sulfur compounds are exemplified with hydrogen sulfide, salts of hydrogen sulfide, salts of sulfide, salts of hydrogen sulfite, salts of sulfite, salts of thiosulfate, salts of thiocyanate, and other inorganic compounds containing sulfur (valence state II or IV).

Among these, more preferred inorganic compounds include; inorganic chloride salts, inorganic bromide salts, and inorganic iodide salts.

Preferred reducing substances also include organic compounds such as: organic chloride salts, organic bromide salts, organic iodide salts, substituted and unsubstituted aromatic hydrocarbons, substituted and unsubstituted heteroaromatic compounds, substituted and unsubstituted unsaturated aliphatic hydrocarbons, substituted and unsubstituted nitrogen-containing aliphatic hydrocarbons, organic sulfur compounds, organic selenium compounds, organic phosphorous compounds, salts or complexes of substituted or unsubstituted heteroaromatic compounds and hydrogen fluoride (HF), salts or complexes of substituted or unsubstituted nitrogen-containing aliphatic hydrocarbons and hydrogen fluoride (HF), and so on.

Preferred organic chloride salts are exemplified with methylammonium chloride, dimethylammonium chloride, trimethylammonium chloride, tetramethylammonium chloride, ethylammonium chloride, diethylammonium chloride, triethylammonium chloride, tetraethylammonium chloride, propylammonium chloride, tripropylammonium chloride, tetrapropylammonium chloride, butylammonium chloride, tributylammonium chloride, tetrabutylammonium chloride, anilinium chloride, N,N-dimethylanilinium chloride, pyridinium chloride, N-methylpyridinium chloride, pyrrolidinium chloride, piperidinium chloride, and other organic salts containing chloride anions.

Preferred organic bromide salts are exemplified with methylammonium bromide, dimethylammonium bromide, trimethylammonium bromide, tetramethylammonium bromide, triethylammonium bromide, tetraethylammonium bromide, tripropylammonium bromide, tributylammonium bromide, tetrabutylammonium bromide, pyridinium bromide, and other organic salts containing bromide anions.

Preferred organic iodide salts are exemplified with methylammonium iodide, dimethylammonium iodide, trimethylammonium iodide, tetramethylammonium iodide, triethylammonium iodide, tetraethylammonium iodide, tributylammonium iodide, tetrabutylammonium iodide, pyridinium iodide, and other organic salts containing iodide anions.

Preferred substituted and unsubstituted aromatic hydrocarbons are exemplified with benzene, toluene, xylene, mesitylene, durene, hexamethylbenzene, anisole, dimethoxybenzene, aniline, N,N-dimethylaniline, phenylenediamine, phenol, salts of phenol, hydrobenzoquinone, naphthalene, indene, anthracene, phenanthrene, pyrene, and so on.

Preferred substituted and unsubstituted heteroaromatic compounds are exemplified with pyridine, methylpyridine, dimethylpyridine, trimethylpyridine, fluoropyridine, chloropyridine, dichloropyridine, pyrrole, indole, quinoline, isoquinoline, carbazole, imidazole, pyrimidine, pyridazine, pyrazine, triazole, furan, benzofuran, thiophene, benzothiophene, thiazole, phenothiazine, phenoxazine, and so on.

Preferred substituted and unsubstituted unsaturated aliphatic hydrocarbons are exemplified with; substituted and unsubstituted alkenes such as ethylene, propene, butene, isobutylene, 2-methyl-2-butene, 2,3-dimethyl-2-butene, 2,3-dimethyl-1-butene, butadiene, pentene, 2-methyl-1-pentene, 2-methyl-2-pentene, hexene, cyclohexene, 1-methyl-1-cyclohexene, 1,2-dimethyl-1-cyclohexene, 2-N,N-diethylamino-1-propene, 1-N,N-dimethylamino-1-cyclohexene, 1-N,N-diethylamino-1-cyclohexene, 1-pyrrolidino-1-cyclohexene, 1-pyrrolidino-1-cyclopentene, styrene, α- and β-methylstyrene, stilbene, 2-methoxy-1-propene (methyl 2-propenyl ether), ethyl vinyl ether, 2,3-dihydrofuran, 2,3-dihydro-5-methylfuran, 3,4-dihydro-2H-pyran, and so on; and substituted and unsubstituted alkynes such as acetylene, propyne, phenylacetylene, diphenylacetylene, and so on.

Preferred substituted and unsubstituted nitrogen-containing aliphatic hydrocarbons are exemplified with methylamine, ethylamine, diethylamine, triethylamine, propylamine, butylamine, pyrrolidine, N-methylpyrrolidine, piperidine, N-methylpiperidine, morpholine, N-methylmorpholine, ethylenediamine, N,N,N′N′-tetramethylethylenediamine, triethylenediamine, urea, tetramethylurea, and so on.

Preferred salts or complexes of substituted and unsubstituted heteroaromatic compounds and hydrogen fluoride (HF), are exemplified with pyridine. HF, pyridine. 2HF, pyridine. 3HF, methylpyridine.HF, dimethylpyridine.HF, trimethylpyridine.HF, and so on.

Preferred salts or complexes of nitrogen-containing aliphatic hydrocarbons and hydrogen fluoride (HF), are exemplified with triethylamine.HF, triethylamine.2HF, triethylamine.3HF, triethylamine.4HF, triethylamine.5HF, trimethylamine.HF, and so on.

Preferred organic sulfur compounds are exemplified with organic sulfides, organic disulfides, organic polysulfides, organic sulfenyl halides, and organic thiols and their salts.

Preferred organic sulfides are exemplified with dimethyl sulfide, diethyl sulfide, dipropyl sulfide, dibutyl sulfide, di(tert-butyl) sulfide, tetrahydrothiophene, methyl phenyl sulfide, trimethylsilyl phenyl sulfide, diphenyl sulfide, bis(o, m, and p-methylphenyl) sulfides, bis(o, m, and p-ethylphenyl) sulfide, bis(o, m, and p-n-propylphenyl) sulfide, bis(o, m, and p-isopropylphenyl) sulfide, bis(o, m, and p-butylphenyl) sulfide, bis(o, m, and p-isobutylphenyl) sulfide, bis(o, m, and p-sec-butylphenyl) sulfide, bis(o, m, and p-tert-butylphenyl) sulfide, each isomer of bis(dimethylphenyl) sulfide, each isomer of bis(trimethylphenyl) sulfide, bis(4-tert-butyl-2,6-dimethylphenyl) sulfide, bis(o, m, and p-fluorophenyl) sulfides, bis(o, m, and p-chlorophenyl) sulfide, bis(o, m, and p-bromophenyl) sulfide, bis(o, m, and p-iodophenyl) sulfides, bis(o, m, and p-nitrophenyl) sulfide, and so on.

Preferred organic disulfides are exemplified with dimethyl disulfide, diethyl disulfide, diisopropyl disulfide, di(tert-butyl) disulfide, diphenyl disulfide, bis(o, m, and p-methylphenyl) disulfides, bis(o, m, and p-ethylphenyl) disulfide, bis(o, m, and p-n-propylphenyl) disulfide, bis(o, m, and p-isopropylphenyl) disulfide, bis(o, m, and p-butylphenyl) disulfide, bis(o, m, and p-isobutylphenyl) disulfide, bis(o, m, and p-sec-butylphenyl) disulfide, bis(o, m, and p-tert-butylphenyl) disulfide, each isomer of bis(dimethylphenyl) disulfide, each isomer of bis(trimethylphenyl) disulfide, bis(4-tert-butyl-2,6-dimethylphenyl) disulfide, bis(o, m, and p-fluorophenyl) disulfides, bis(o, m, and p-chlorophenyl) disulfide, bis(o, m, and p-bromophenyl) disulfide, bis(o, m, and p-iodophenyl) disulfides, bis(o, m, and p-nitrophenyl) disulfide, and so on.

Preferred organic polysulfides are exemplified with diphenyl trisulfide, dimethyl trisulfide, and so on.

Preferred organic sulfenyl halides are exemplified with phenylsulfenyl fluoride, phenylsulfenyl chloride, phenylsulfenyl bromide, phenylsulfenyl iodide, phenylsufinyl chloride, o, m, and p-methylphenylsulfenyl chloride, o, m, and p-ethylphenylsulfenyl chloride, o, m, and p-n-propylphenylsulfenyl chloride, o, m, and p-isopropylphenylsulfenyl chloride, o, m, and p-butylphenylsulfenyl chloride, o, m, and p-isobutylphenylsulfenyl chloride, o, m, and p-sec-butylphenylsulfenyl chloride, o, m, and p-tert-butylphenylsulfenyl chloride, 2,4-dimethylphenylsulfenyl chloride, 2,5-dimethylphenylsulfenyl chloride, 2,4,6-trimethylphenylsulfenyl chloride, 4-tert-butyl-2,6-dimethylphenylsulfenyl chloride, o, m, and p-fluorophenylsulfenyl chloride, o, m, and p-chlorophenylsulfenyl chloride, o, m, and p-bromophenylsulfenyl chloride, o, m, and p-iodophenylsulfenyl chloride, o, m, and p-nitrophenylsulfenyl chloride, and so on.

Preferred organic thiols and their salts are exemplified with methanethiol, ethanethiol, propanethiol, isopropanethiol, butanethiol, sec-butanethiol, isobutanethiol, tert-butanethiol, thiophenol, o, m, and p-methylbenzenethiols, o, m, and p-ethylbenzenethiol, o, m, and p-n-propylbenzenethiol, o, m, and p-isopropylbenzenethiol, o, m, and p-butylbenzenethiol, o, m, and p-isobutylbenzenethiol, o, m, and p-sec-butylbenzenethiol, o, m, and p-tert-butylbenzenethiol, each isomers of dimethylbenzenethiol, each isomer of trimethylbenzenethiol, 4-tert-butyl-2,6-dimethylbenzenethiol, o, m, and p-chlorobenzenethiols, o, m, and p-fluorobenzenethiols, o, m, and p-bromobenzenethiols, o, m, and p-iodobenzenethiol, o, m, and p-nitrobenzenethiol, and metal salts, ammonium salts, phosphonium salts of these organic thiols.

Preferred organic selenium compounds are exemplified with benzeneselenol, diphenyl selenide, diphenyl diselenide, and so on.

Preferred organic phosphorous compounds are exemplified with trimethylphosphine, triethylphosphine, tripropylphosphine, tributylphosphine, triphenylphosphine, trimethylphosphite, triethylphosphite, tripropylphosphite, tributylphosphite, triphenylphosphite, and so on.

Preferred reducing substances in general include; the elements such as alkali metals, alkali earth metals, transition metals, metals in Groups 13˜15 of the Periodic Table, and semi-metals; the inorganic compounds such as inorganic chloride salts, inorganic bromide salts, inorganic iodide salts; and the organic compounds such as organic chloride salts, organic bromide salts, organic iodide salts, substituted and unsubstituted aromatic hydrocarbons, substituted and unsubstituted heteroaromatic compounds, substituted and unsubstituted unsaturated aliphatic hydrocarbons, substituted and unsubstituted nitrogen-containing aliphatic hydrocarbons, organic sulfur compounds, salts or complexes of substituted or unsubstituted heteroaromatic compounds and hydrogen fluoride, and salts or complexes of substituted or unsubstituted nitrogen-containing aliphatic hydrocarbons and hydrogen fluoride.

Arylsulfur halotetrafluoride of the formula (I) may be derived by an existing reducing substance to another compound(s) that can more effectively fluorinate a target compound. The compound(s) includes any derived compounds that can fluorinate a target compound. A preferable derived compound is an arylsulfur trifluoride represented by the formula (II) shown below.

In one embodiment, preferred reducing substance herein is a substance which reduces arylsulfur halotetrafluoride of the formula (I) to arylsulfur trifluoride represented by the formula (II):

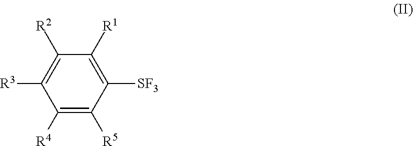

in which R¹, R², R³, R⁴, and R⁵ are the same as described above.

A further preferred reducing substance is a substance which reduces arylsulfur halotetrafluoride of the formula (I) to arylsulfur trifluoride represented by the formula (II) in high yields without reducing or with limited reducing arylsulfur trifluoride represented by the formula (II).

In this aspect, reducing substances include the same reducing substances as above. Preferred reducing substances include; the elements such as alkali metals, alkali earth metals, transition metals, metals in Groups 13˜15 of the Periodic Table, and semi-metals; inorganic compounds such as inorganic chloride salts, inorganic bromide salts, inorganic iodide salts; and the organic compounds such as organic chloride salts, organic bromide salts, organic iodide salts, substituted and unsubstituted aromatic hydrocarbons, substituted and unsubstituted heteroaromatic compounds, substituted and unsubstituted unsaturated aliphatic hydrocarbons, substituted and unsubstituted nitrogen-containing aliphatic hydrocarbons, organic sulfur compounds, salts or complexes of substituted or unsubstituted heteroaromatic compounds and hydrogen fluoride, and salts or complexes of substituted or unsubstituted nitrogen-containing aliphatic hydrocarbons and hydrogen fluoride; and mixtures thereof.

As more preferred reducing substances, there are exemplified alkali metals, alkali earth metals, transition metals, metals in Groups 13˜15 of the Periodic Table, semi-metals, inorganic chloride salts, inorganic bromide salts, inorganic iodide salts, organic bromide salts, organic iodide salts, substituted and unsubstituted aromatic hydrocarbons, substituted and unsubstituted heteroaromatic compounds, substituted and unsubstituted unsaturated aliphatic hydrocarbons, substituted and unsubstituted nitrogen-containing aliphatic hydrocarbons, organic sulfur compounds, salts or complexes of substituted or unsubstituted heteroaromatic compounds and hydrogen fluoride, and salts or complexes of substituted or unsubstituted nitrogen-containing aliphatic hydrocarbons and hydrogen fluoride, and mixtures thereof.

The amount used of reducing substance is dependent on the kind and nature of the reducing substance and reaction conditions such as temperature and solvent in addition to arylsulfur halotetrafluorides. Therefore, one can choose the amount necessary for obtaining an adequate yield of the fluorinated compound in each reaction. An illustrative amount used of reducing substance per one mole of an arylsulfur halotetrafluoride is from about 0.1 moles to about ten moles and more typically about 0.1 moles to about five moles.

The amount used of arylsulfur halotetrafluoride is dependent on the kind and nature of the target compound or reducing substance and reaction conditions such as temperature and solvent. Therefore, one can choose the amount necessary for obtaining an adequate yield of the fluorinated compound from the target compound in each reaction. An illustrative amount used of arylsulfur halotetrafluoride per one mole of a target compound is from about 0.5 moles to about ten moles and more typically about one mole to about five moles.

In some cases, fluorination is conducted in the presence of an acid such as a Brönsted acid or a Lewis acid in order to accelerate reaction or to increase fluorination yields. The amount of acid used in any given reaction depends on the target compound(s), other chemical compounds and/or reaction conditions. Preferably, the Brönsted acid is at least one member selected from a group consisting of hydrogen fluoride (HF), HBF₄, HBCl₄, HBFCl₃, HSbF₄, HSbFCl₃, HSbF₆, HSbFCl₅, HSbF₄Cl₂, HN(SO₂CF₃)₂, and other like acids, and their complexes with organic compounds such as ethers and amines. Complexes of HF with amines are preferably illustrated as mixtures of HF and pyridine, a mixture of HF and α, β, and/or γ-methylpyridine, a mixture of HF and dimethylpyridine, a mixture of HF and trimethylpyridine, a mixture of HF and trimethylamine, a mixture of HF and triethylamine, and so on. Among complexes with amines, an about 70:30 wt % mixture of HF and pyridine, a 3:1 molar ratio mixture of HF and triethylamine, and a 5:1 molar ratio mixture of HF and triethylamine are more preferable because of availability. Preferably, the Lewis acid is at least one member selected from a group consisting of BF₃, BCl₃, SbF₃, SbCl₃, SbF₆, SbCl₆, SbF₃Cl₂, SnCl₄, SnF₄, SnCl₃F, TiF₄, TiCl₄, and other like acids, and their complexes with organic compounds such as ethers, nitriles, and so on. As the preferred complexes, BF₃ etherates can be exemplified.

In some cases, fluorination is conducted in the presence of a base in order to increase production yields, or when starting materials and/or products are sensitive to acid conditions. Preferable bases are exemplified by metal fluorides such as sodium fluoride, potassium fluoride, cesium fluoride, and other like compounds; carbonates such as sodium carbonate, sodium bicarbonate, potassium carbonate, potassium bicarbonate, and other like compounds. The amount of base used in any given reaction depends on the target compound(s), other chemical compounds and/or reaction conditions.

The fluorination is typically conducted in the presence or absence of a solvent. In some cases, reaction can be preferably conducted without a solvent. In other cases, solvent is used for mild or selective fluorination, and solvent is preferably selected from a group consisting of hydrocarbons, halocarbons, ethers, nitriles, aromatics, nitro compounds, esters, and mixture thereof. These solvents are exemplified as described above.

Preferably, reaction temperature for the fluorination can be selected from the range of about −80° C. to about +200° C., and more preferably, about −50° C. to about +150° C. The reaction temperature is dependent on the arylsulfur halotetrafluorides, reducing substances, target compounds, solvents, and catalysts or additives used. Therefore, one can choose the temperature necessary for the reaction.

The reaction time is also dependent on reaction temperature, arylsulfur halotetrafluorides, reducing substances, target compounds, solvents, catalysts or additives, and their amounts used. Therefore, one can choose the time necessary for completing each reaction by modifying one or more of these parameters, but can be from about 1 minute to several days, and is preferably within a few days.

In another embodiment, a two-step process is provided for introducing one or more fluorine atoms into a target compound comprising (step 1) contacting arylsulfur halotetrafluoride represented by the formula (I) shown above with a reducing substance that reduces the arylsulfur halotetrafluoride; and (step 2) contacting a target compound with the resulting mixture from step 1.

This method consists of two processes; the first step being a reaction of an arylsulfur halotetrafluoride with a reducing substance, and the second step fluorination of a target compound with the resulting mixture.

In the first step, arylsulfur halotetrafluoride of the formula (I) may be derived by a reducing substance to another compound(s) that can more effectively fluorinate a target compound. The compound(s) includes any derived compounds that can fluorinate a target compound. A preferable derived compound is an arylsulfur trifluoride represented by the formula (II) shown below.

A preferred reducing substance in this two-step reaction is a substance which reduces arylsulfur halotetrafluoride of the formula (I) to arylsulfur trifluoride represented by formula (II):

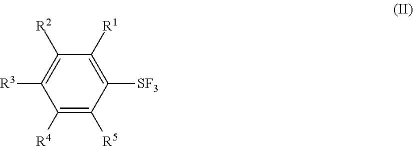

in which R¹, R², R³, R⁴, and R⁵ are the same as previously described.

In this alternative aspect, the method consists of two processes; the first step is a reaction of an arylsulfur halotetrafluoride with a reducing substance to form an arylsulfur trifluoride; the second is the fluorination of a target compound with the reaction mixture obtained from the first step.

The arylsulfur halotetrafluorides are described as above.

Reducing substances usable in this embodiment are the same as described above. The amount used of reducing substance is dependent on the kind and nature of the reducing substance and reaction conditions such as temperature and solvent in addition to arylsulfur halotetrafluorides. Therefore, one can choose the amount necessary for obtaining an adequate yield of the fluorinated compound in the second fluorination step. An illustrative amount used of reducing substance per one mole of an arylsulfur halotetrafluoride is from about 0.1 moles to about ten moles and more typically about 0.1 moles to about five moles.

The first step reaction is conducted in the present or absence of a solvent. In some cases, reactions can be preferably conducted without a solvent. In some cases, solvent is used for mild or selective fluorination, and solvent is preferably selected from a group consisting of hydrocarbons, halocarbons, ethers, nitriles, aromatics, nitro compounds, esters, and mixture thereof. These solvents are exemplified as described above.

The reaction temperature can be selected in the range of about −80° C. to about +200° C., and more preferably, about −50° C. to about +150° C. The reaction temperature is dependent on the arylsulfur halotetrafluorides, reducing substances, and solvents used. Therefore, one can choose the temperature necessary for the reaction.

The reaction time is also dependent on reaction temperature, arylsulfur halotetrafluorides, reducing substances, solvents, and their amounts used. Therefore, one can choose the time necessary for completing each reaction by modifying one or more these parameters, but can be from about 1 minute to several days, preferably, within a few days.

For the second fluorination step, the target compounds are described above.

In some cases, fluorination is conducted in the presence of an acid such as a Brönsted acid or a Lewis acid in order to accelerate reaction or to increase fluorination yields. The amount of acid used in any given reaction depends on the target compound(s), other chemical compounds and/or reaction conditions. Preferably, the Brönsted acid is at least one member selected from a group consisting of hydrogen fluoride (HF), HBF₄, HBCl₄, HBFCl₃, HSbF₄, HSbFCl₃, HSbF₆, HSbFCl₅, HSbF₄Cl₂, HN(SO₂CF₃)₂, and other like acids, and their complexes with organic compounds such as ethers and amines. Illustrative complexes of HF with amines include: a mixture of HF and pyridine, a mixture of HF and α, β, and/or γ-methylpyridine, a mixture of HF and dimethylpyridine, a mixture of HF and trimethylpyridine, a mixture of HF and trimethylamine, a mixture of HF and triethylamine, and so on. Among complexes with amines, an about 70:30 wt % mixture of HF and pyridine, a 3:1 molar ratio mixture of HF and triethylamine, and a 5:1 molar ratio mixture of HF and triethylamine are more preferable because of availability. Preferably, the Lewis acid is at least one member selected from a group consisting of BF₃, BCl₃, SbF₃, SbCl₃, SbF₆, SbCl₆, SbF₃Cl₂, SnCl₄, SnF₄, SnCl₃F, TiF₄, TiCl₄, and other like acids, and their complexes with organic compounds such as ethers, nitriles, and so on. As the preferred complexes, BF₃ etherates can be exemplified.

In some cases, fluorination is conducted in the presence of a base in order to increase product yields, or when starting materials and/or products are sensitive to acid conditions. Preferable bases are exemplified with metal fluorides such as sodium fluoride, potassium fluoride, cesium fluoride, and other like compounds; carbonates such as sodium carbonate, sodium bicarbonate, potassium carbonate, potassium bicarbonate, and other like compounds; amines such as pyridine, chloropyridine, fluoropyridine, methylpyridine, dimethylpyridine, trimethylpyridine, diethylamine, triethylamine, and other like compounds. The amount of base used in any given reaction depends on the target compound(s), other chemical compounds and/or reaction conditions.

The amount used of target compound is dependent on the kind and nature of the target compound and the arylsulfur halotetrafluoride and reducing substance of the first step and reaction conditions such as temperature, solvent, and catalyst or additive used. Therefore, one can choose the amount necessary for obtaining an adequate yield of the fluorinated compound from the target compound in each reaction. An illustrative amount used of target compound per one mole of an arylsulfur halotetrafluoride used in the first step is from about 0.1 moles to about 2 moles and more typically about 0.2 moles to about one mole.

The fluorination is conducted in the presence or absence of a solvent. In some cases, reactions can be preferably conducted without a solvent. In some cases, solvent is used for mild or selective fluorination, and solvent is preferably selected from a group consisting of hydrocarbons, halocarbons, ethers, nitriles, aromatics, nitro compounds, esters, and mixture thereof. These solvents are exemplified as described above.

Reaction temperature of the fluorination step can be selected from the range of about −80° C. to about +200° C., and more preferably, about −50° C. to about +150° C. The reaction temperature is dependent on the target compounds, arylsulfur halotetrafluorides and reducing substances of the first step, solvents, and catalysts or additives used. Therefore, one can choose the temperature necessary for the reaction.

The reaction time is also dependent on reaction temperature, target compounds, arylsulfur halotetrafluorides and reducing substances of the first step, solvents, catalysts or additives, and their amounts used. Therefore, one can choose the time necessary for completing each reaction by modifying one or more these parameters, but can be from about 1 minute to several days, preferably within a few days.

In one embodiment, a process of introducing one or more fluorine atoms into a target compound comprises: (step 1) contacting arylsulfur halotetrafluoride represented by the formula (I) with a reducing substance to form arylsulfur trifluoride represented by the formula (II), and then (step 2) contacting a target compound with the arylsulfur trifluoride obtained from the first step.

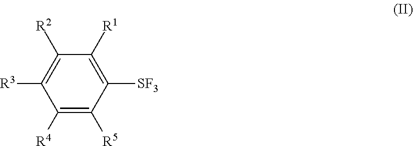

in which R¹, R², R³, R⁴, and R⁵ are the same as above.

This method consists of two processes; the first step is a reaction of an arylsulfur halotetrafluoride with a reducing substance, and the second step is the fluorination of a target compound with the arylsulfur trifluoride obtained from the first step. The arylsulfur trifluoride may be isolated and used for the next fluorination (step 2) or may be used without isolation for the next step 2. The use without isolation is preferable because arylsulfur trifluorides are sensitive to moisture or wet conditions.

The arylsulfur halotetrafluorides of formula (I) are described as above.

The R¹, R², R³, R⁴, and R⁵ of the arylsulfur trifluorides (products) represented by the formula (II) may be different from the R¹, R², R³, R⁴, and R⁵ of the starting materials represented by the formula (I). Thus, embodiments of this invention include transformation of the R¹, R², R³, R⁴, and R⁵ to another R¹, R², R³, R⁴, and R⁵ which may take place under the reaction conditions or during the reaction of the present invention as long as the SF₄X group is transformed to a SF₃ group.

Reducing substances are described and exemplified above. Preferred reducing substances for this embodiment include any substance which reduces arylsulfur halotetrafluoride of the formula (I) to arylsulfur trifluoride represented by the formula (II) in high yields without reducing or with limited reducing arylsulfur trifluoride represented by the formula (II). Preferable reducing substances include; the elements such as alkali metals, alkali earth metals, transition metals, metals in Groups 13˜15 of the Periodic Table, and semi-metals; the inorganic compounds such as inorganic chloride salts, inorganic bromide salts, inorganic iodide salts; and the organic compounds such as organic chloride salts, organic bromide salts, organic iodide salts, substituted and unsubstituted aromatic hydrocarbons, substituted and unsubstituted heteroaromatic compounds, substituted and unsubstituted unsaturated aliphatic hydrocarbons, substituted and unsubstituted nitrogen-containing aliphatic hydrocarbons, organic sulfur compounds, salts or complexes of substituted or unsubstituted heteroaromatic compounds and hydrogen fluoride, and salts or complexes of substituted or unsubstituted nitrogen-containing aliphatic hydrocarbons and hydrogen fluoride; and mixtures thereof.

As more preferred reducing substances, there are exemplified alkali metals, alkali earth metals, transition metals, metals in Groups 13˜15 of the Periodic Table, semi-metals, inorganic chloride salts, inorganic bromide salts, inorganic iodide salts, organic chloride salts, organic bromide salts, organic iodide salts, substituted and unsubstituted aromatic hydrocarbons, substituted and unsubstituted heteroaromatic compounds, substituted and unsubstituted unsaturated aliphatic hydrocarbons, substituted and unsubstituted nitrogen-containing aliphatic hydrocarbons, organic sulfur compounds, salts or complexes of substituted or unsubstituted heteroaromatic compounds and hydrogen fluoride, and salts or complexes of substituted or unsubstituted nitrogen-containing aliphatic hydrocarbons and hydrogen fluoride, and mixtures thereof.

In addition, among the reducing substances, inorganic chloride salts, inorganic bromide salts, inorganic iodide salts, organic chloride salts, organic bromide salts, organic iodide salts, organic sulfides, organic disulfides, organic thiols or their salts, organic sulfenyl halides, substituted and unsubstituted heteroaromatic compounds, and substituted and unsubstituted unsaturated aliphatic hydrocarbons, are more preferable. Among the chloride, bromide, and iodide salts, inorganic chloride salts are more preferable, and additionally, among inorganic chloride salts, alkali metal chlorides such as LiCl, NaCl, KCl, RbCl, and CsCl are more preferable. Among these, LiCl, NaCl, KCl, and CsCl are more preferable, and KCl is furthermore preferable because of cost and high yield reactions. Among substituted and unsubstituted heteroaromatic compounds, pyridine and its derivatives such as methylpyridine, dimethylpyridine, trimethylpyridine, fluoropyridine, chloropyridine, dichloropyridine, and so on, are preferable because of cost and mild and high yield reactions. Among substituted and unsubstituted unsaturated aliphatic hydrocarbons, alkyl alkenyl ethers such as 2-methoxy-1-propene (methyl 2-propenyl ether), ethyl vinyl ether, 2,3-dihydrofuran, 2,3-dihydro-5-methylfuran, 3,4-dihydro-2H-pyran, and so on, are more preferable because of high yield reactions.

Other furthermore preferred reducing substances include arylsulfur compounds having a formula (IIIa) and/or a formula (IIIb) as shown below. There are two advantages to these compounds: (1) in most cases, products other than arylsulfur trifluorides can be gaseous compounds such as chlorine gas (Cl₂), which is easily removed from the arylsulfur trifluorides that are liquid or solid, and (2) an arylsulfur trifluoride or a total amount of arylsulfur trifluorides can be prepared in a more molar amount than that of arylsulfur halotetrafluoride used as a starting material, as seen in Eqs 1˜4 and Examples 71˜74 below. The arylsulfur trifluorides obtained can be used for the second step reaction.

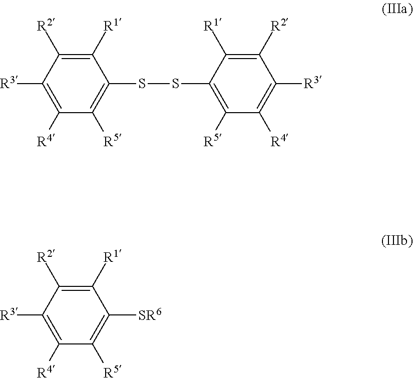

wherein R^{1′}, R^{2′}, R^{3′}, R^{4′}, and R^{5′} are the same as R¹, R², R³, R⁴, and R⁵ above, and R⁶ is a hydrogen atom, a halogen atom, a metal atom, an ammonium moiety, a phosphonium moiety, or a silyl moiety.

The halogen atom of R⁶ is a fluorine, chlorine, bromine, or iodine atom. Among them, a chlorine atom is preferred in viewpoint of cost. As a metal salt, alkali metals, alkali earth metals, and transition metals are exemplified, and among them, alkali metals such as Li, Na, Ka, and Cs are preferably exemplified. As an ammonium moiety, ammonium, trimethylammonium, triethylammonium, tetramethylammonium, tetraethylammonium, tetrabutylammonium, benzyldimethylammonium, and pyridinium are preferably exemplified. As a phosphonium moiety, tetraphenylphosphonium is preferably exemplified. As a silyl moiety, trimethylsilyl, tert-butyldimethylsilyl, triethylsilyl, tripropylsilyl, and tributylsilyl are preferably exemplified.

When Ar═Ar′, the reaction of arylsulfur halotetrafluoride of formula (I) (represented as ArSF₄X) and a reducing substance (IIIa) (represented as Ar′SSAr′) can give a single product (ArSF₃) as represented by the formula (II). When Ar≠Ar′, the reaction of (I) and (IIIa) provides a product of a mixture of (II) and (II′) shown below. Formula (II′) is represented as Ar′SF₃. Both ArSF₃ and Ar′SF₃ can be used for the second step fluorination process.

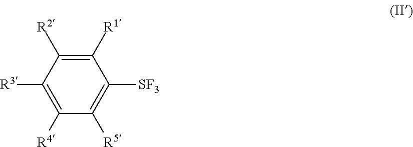

Similarly, when Ar═Ar′, the reaction of arylsulfur halotetrafluoride (I) (represented as ArSF₄X) and a reducing substance (IIIb) (represented as Ar′SR⁶) can give a single product (ArSF₃) as represented by the formula (II). When Ar≠Ar′, the reaction of (I) and (IIIb) provides a product of a mixture of (II) and (II′).

When arylsulfur compound having a formula (IIIa) (Ar′SSAr′) is used, the reaction equation of the reaction of (I) and (IIIa) may be shown with the following:

ArSF₄X+⅙.Ar′SSAr′→ArSF₃+⅓.Ar′SF₃+½.X₂ (Eq. 1)

Thus, an illustrative total amount of arylsulfur trifluorides (ArSF₃ and Ar′SF₃) is 4/3 moles for every one mole of ArSF₄X used.

When arylsulfur compound having a formula (IIIb) (Ar′SR⁶) is used, the reaction equation of reaction of (I) and (IIIb) may be the following:

1, R⁶≠a halogen atom;

ArSF₄X+¼.Ar′SR⁶→ArSF₃+¼.Ar′SF₃+½.X₂+¼.R⁶F (Eq. 2)
Thus, an illustrative total amount of arylsulfur trifluorides (ArSF₃ and Ar′SF₃) is 5/4 moles for every one mole of ArSF₄X used.

2, R⁶═X (a chlorine, bromine, or iodine atom);

ArSF₄X+⅓.Ar′SX→ArSF₃+⅓.Ar′SF₃+⅔.X₂ (Eq. 3)
Thus, an illustrative total amount of arylsulfur trifluorides is 4/3 moles for every one mole of ArSF₄X used.

3, R⁶=a fluorine atom;

ArSF₄X+½.Ar′SF→ArSF₃+½.Ar′SF₃+½.X₂ (Eq. 4)

Thus, an illustrative total amount of arylsulfur trifluorides is 3/2 moles for every one mole of ArSF₄X used.

The R^{1′}, R^{2′}, R^{3′}, R^{4′}, and R^{5′} of the products (Ar′SF₃) represented by the formula (II′) may be different from the R^{1′}, R^{2′}, R^{3′}, R^{4′}, and R^{5′} of the starting materials represented by the formulas (IIIa) and/or (IIIb). Thus, embodiments of this invention include transformation of the R^{1′}, R^{2′}, R^{3′}, R^{4′}, and R^{5′} to another R^{1′}, R^{2′}, R^{3′}, R^{4′}, and R^{5′} which may take place during the reaction of the present invention or under the reaction conditions as long as the —S—S— or —S— moiety is transformed to a —SF₃ group(s).

In order to get adequate production yields, the amount used of arylsulfur compound having a formula (IIIa) is around ⅙ moles per one mole of ArSF₄X. The amount used of arylsulfur compound having a formula (IIIb) is around 1/4 moles per one mole of ArSF₄X when R₆≠a halogen atom. The amount used of arylsulfur compound having a formula (IIIb) is around ⅓ moles per one mole of ArSF₄X when R⁶═Cl, Br, or I. The amount used of arylsulfur compound having a formula (IIIb) is around ½ moles per one mole of ArSF₄X when R⁶═F. Other amounts for each reaction can be used, but the above numbers provide relative levels that translate into adequate production yields.

The amount used of reducing substance other than the arylsulfur compound (IIIa) or (IIIb) can be selected for each reaction to obtain an adequate yield of arylsulfur trifluoride because the necessary amount of reducing substance is dependent on the kind and nature of the reducing substance and reaction conditions such as temperature and solvent in addition to arylsulfur halotetrafluorides. An illustrative amount used of reducing substance per one mole of an arylsulfur halotetrafluoride is from about 0.1 moles to about ten moles and more typically about 0.1 moles to about five moles.

The first step reaction is conducted in the present or absence of a solvent. In some cases, reactions can be preferably conducted without a solvent. In other cases, solvent is used for mild or selective fluorination, and solvent is preferably selected from a group consisting of hydrocarbons, halocarbons, ethers, nitriles, aromatics, nitro compounds, esters, and mixture thereof. These solvents are exemplified as described above.

The reaction temperature of the first step reaction can be selected in the range of about −80° C. to about +200° C., and more preferably, about −50° C. to about +150° C. The reaction temperature primarily depends on the arylsulfur halotetrafluorides, reducing substances, and solvents used. Therefore, one can choose the temperature necessary for the reaction. When arylsulfur compounds having a formula (IIIa) or a formula (IIIb) (R⁶=a halogen atom) are used as a reducing substance, the reaction temperature is preferably selected from the range of about room temperature to about +150° C., and more preferably, about +50° C. to about +120° C. When arylsulfur compounds having a formula (IIIb) (R⁶≠a halogen atom) are used as a reducing substance, the reaction temperature can be preferably selected from the range of about −80° C. to about +150° C., and more preferably, about −50° C. to about +120° C. When inorganic or organic chloride salts are used as a reducing substance, the reaction temperature is preferably selected from the range of about +40° C. to about +150° C., more preferably, about +50° C. to about +120° C. When heteroaromatic compounds are used as a reducing substance, the reaction temperature is preferably selected in the range of about −50° C. to about +100° C., preferably, about −20° C. to about +70° C. When other reducing substances are used, the reaction temperature can be selected so that the reaction is finished in a reasonable time.

The reaction time is also dependent on reaction temperature, reducing substances, arylsulfur halotetrafluorides, solvents, and their amounts used. Therefore, one can choose the time necessary for completing each reaction by modifying one or more of these parameters, but can be from about 1 minute to several days, preferably, within a few days.

For the second fluorination step, the arylsulfur trifluorides obtained by the first step may be used without isolation or the isolated arylsulfur trifluorides may be used. It is preferable that the arylsulfur trifluorides are used without isolation because it is convenient since the arylsulfur trifluorides are sensitive to moisture or wet conditions.

For the second fluorination step, the target compounds are the same as described above.

In some cases, fluorination is conducted in the presence of an acid such as a Brönsted acid or a Lewis acid in order to accelerate reaction or to increase fluorination yields. The amount of acid used in any given reaction depends on the target compound(s), other chemical compounds and/or reaction conditions. Preferably, the Brönsted acid is at least one member selected from a group consisting of hydrogen fluoride (HF), HBF₄, HBCl₄, HBFCl₃, HSbF₄, HSbFCl₃, HSbF₆, HSbFCl₅, HSbF₄Cl₂, HN(SO₂CF₃)₂, and other like acids, and their complexes with organic compounds such as ethers and amines Illustrative complexes of HF with amines, include a mixture of HF and pyridine, a mixture of HF and α, β, and/or γ-methylpyridine, a mixture of HF and dimethylpyridine, a mixture of HF and trimethylpyridine, a mixture of HF and trimethylamine, a mixture of HF and triethylamine, and so on. Among complexes with amines, an about 70:30 wt % mixture of HF and pyridine, a 3:1 molar ratio mixture of HF and triethylamine, and a 5:1 molar ratio mixture of HF and triethylamine are more preferable because of availability. Preferably, the Lewis acid is at least one member selected from a group consisting of BF₃, BCl₃, SbF₃, SbCl₃, SbF₆, SbCl₆, SbF₃Cl₂, SnCl₄, SnF₄, SnCl₃F, TiF₄, TiCl₄, and other like acids, and their complexes with organic compounds such as ethers, nitriles, and so on. As the preferred complexes, BF₃ etherates are exemplified.

In some cases, fluorination is conducted in the presence of a base in order to increase production yields, or when starting materials and/or products are sensitive to acid conditions. Preferable bases are exemplified with metal fluorides such as sodium fluoride, potassium fluoride, cesium fluoride, and other like compounds; carbonates such as sodium carbonate, sodium bicarbonate, potassium carbonate, potassium bicarbonate, and other like compounds; amines such as pyridine, chloropyridine, fluoropyridine, methylpyridine, dimethylpyridine, trimethylpyridine, diethylamine, triethylamine, and other like compounds. The amount of base used in any given reaction depends on the target compound(s), other chemical compounds and/or reaction conditions.

The amount used of target compound is dependent on the kind and nature of the target compound and reaction conditions such as temperature, solvent, and catalyst or additive used. Therefore, one can choose the amount necessary for obtaining an adequate yield of the fluorinated compound from the target compound in each reaction. An illustrative amount used of target compound per one mole of an arylsulfur trifluoride obtained in the first step is from about 0.1 moles to about two moles and more typically about 0.2 moles to about one mole.

The fluorination reaction can be conducted in the presence or absence of a solvent. In some cases, reactions are conducted without a solvent. In other cases, a solvent can be used for mild or selective fluorination, and solvent is preferably selected from a group consisting of hydrocarbons, halocarbons, ethers, nitriles, aromatics, nitro compounds, esters, and mixture thereof. These solvents are exemplified as described above.

Preferably, reaction temperature for the fluorination can be selected in the range of about −80° C. to about +200° C., and more preferably, about −50° C. to about +150° C. The reaction temperature primarily depends on the target compounds, the arylsulfur trifluorides, catalysts, additives, solvents, reducing substances, or the arylsulfur halotetrafluorides used. Therefore, one can choose the temperature necessary for the reaction.

The reaction time is also dependent on reaction temperature, target compounds, arylsulfur trifluorides or halotetrafluorides, solvents, catalysts or additives, and their amounts used. Therefore, one can choose the time necessary for completing each reaction by modifying one or more these parameters, but can be from about 1 minute to several days, preferably within a few days.

The present invention also provides a process for preparing arylsulfur trifluoride as represented by formula (II).

In one embodiment, a process for preparing arylsulfur trifluoride represented by the formula (II) comprises contacting arylsulfur halotetrafluoride represented by the formula (I) above with a reducing substance.

Reducing substances are described and exemplified as above. Preferred reducing substances include any substance which reduces arylsulfur halotetrafluoride of the formula (I) to produce arylsulfur trifluoride represented by the formula (II) in high yields without reducing or with limited reducing arylsulfur trifluoride represented by the formula (II). Preferable reducing substances include; the elements such as alkali metals, alkali earth metals, transition metals, metals in Groups 13˜15 of the Periodic Table, and semi-metals; the inorganic compounds such as inorganic chloride salts, inorganic bromide salts, inorganic iodide salts; and the organic compounds such as organic chloride salts, organic bromide salts, organic iodide salts, substituted and unsubstituted aromatic hydrocarbons, substituted and unsubstituted heteroaromatic compounds, substituted and unsubstituted unsaturated aliphatic hydrocarbons, substituted and unsubstituted nitrogen-containing aliphatic hydrocarbons, organic sulfur compounds, salts or complexes of substituted or unsubstituted heteroaromatic compounds and hydrogen fluoride, and salts or complexes of substituted or unsubstituted nitrogen-containing aliphatic hydrocarbons and hydrogen fluoride; and mixtures thereof.

As more preferred reducing substances, there are exemplified alkali metals, alkali earth metals, transition metals, metals in Groups 13˜15 of the Periodic Table, semi-metals, inorganic chloride salts, inorganic bromide salts, inorganic iodide salts, organic chloride salts, organic bromide salts, organic iodide salts, substituted and unsubstituted aromatic hydrocarbons, substituted and unsubstituted heteroaromatic compounds, substituted and unsubstituted unsaturated aliphatic hydrocarbons, substituted and unsubstituted nitrogen-containing aliphatic hydrocarbons, organic sulfur compounds, salts or complexes of substituted or unsubstituted heteroaromatic compounds and hydrogen fluoride, and salts or complexes of substituted or unsubstituted nitrogen-containing aliphatic hydrocarbons and hydrogen fluoride, and mixtures thereof.

In addition, among the reducing substances, inorganic chloride salts, inorganic bromide salts, inorganic iodide salts, organic chloride salts, organic bromide salts, organic iodide salts, organic sulfides, organic disulfides, organic thiols or their salts, organic sulfenyl halides, substituted and unsubstituted heteroaromatic compounds, and substituted and unsubstituted unsaturated aliphatic hydrocarbons, are more preferable. Among the chloride, bromide, and iodide salts, inorganic chloride salts are more preferable, and additionally, among inorganic chloride salts, alkali metal chlorides such as LiCl, NaCl, KCl, RbCl, and CsCl are more preferable. Among these, LiCl, NaCl, KCl, and CsCl are more preferable, and KCl is furthermore preferable because of cost and high yield reactions. Among substituted and unsubstituted heteroaromatic compounds, pyridine and its derivatives such as methylpyridine, dimethylpyridine, trimethylpyridine, chloropyridine, dichloropyridine, and so on, are preferable because of cost and mild and high yield reactions. Among substituted and unsubstituted unsaturated aliphatic hydrocarbons, alkyl alkenyl ethers such as 2-methoxy-1-propene (methyl 2-propenyl ether), ethyl vinyl ether, 2,3-dihydrofuran, 2,3-dihydro-5-methylfuran, 3,4-dihydro-2H-pyran, and so on, are more preferable because of high yield reactions.

Other furthermore preferred reducing substances include arylsulfur compounds having a formula (IIIa) and/or a formula (IIIb) as shown below, two advantages to these compounds include: (1) in most cases, products other than arylsulfur trifluorides can be gaseous compounds such as chlorine gas (Cl₂), which is easily removed from the arylsulfur trifluorides that are liquid or solid; and (2) another benefit of the reducing substances is that an arylsulfur trifluoride or a total amount of arylsulfur trifluorides can be prepared in a more molar amount than that of arylsulfur halotetrafluoride used as a starting material, as seen in Eqs 1˜4 above and Examples 71˜74 below. The arylsulfur trifluorides obtained can be used for the fluorination reaction of a target compound.

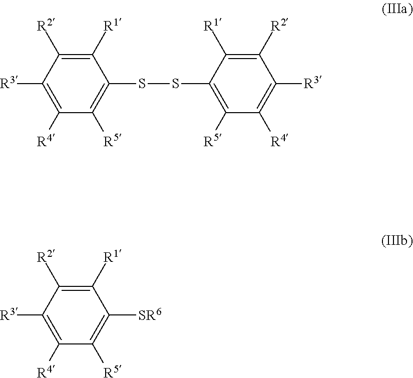

wherein R^{1′}, R^{2′}, R^{3′}, R^{4′}, and R⁵ are the same as R¹, R², R³, R⁴, and R⁵ above, and R⁶ is a hydrogen atom, a halogen atom, a metal atom, an ammonium moiety, a phosphonium moiety, or a silyl moiety.

The halogen atom of R⁶ is a fluorine, chlorine, bromine, or iodine atom. Among them, a chlorine atom is preferred from the viewpoint of cost.

As a metal salt, alkali metals, alkali earth metals, and transition metals are exemplified, and among them, alkali metals such as Li, Na, Ka, and Cs are preferably exemplified. As an ammonium moiety, ammonium, trimethylammonium, triethylammonium, tetramethylammonium, tetraethylammonium, tetrabutylammonium, benzyldimethylammonium, and pyridinium are preferable exemplified. As a phosphonium moiety, tetraphenylphosphonium is preferably exemplified. As a silyl moiety, trimethylsilyl, tert-butyldimethylsilyl, triethylsilyl, tripropylsilyl, and tributylsilyl are preferably exemplified.

When Ar═Ar′, the reaction of (I) (represented as ArSF₄X) and (IIIa) (represented as Ar′SSAr′) can give a single product (ArSF₃) as represented by the formula (II). When Ar≠Ar′, the reaction of (I) and (IIIa) provides a product of a mixture of (II) (ArSF₃) and (II′) shown below. (II′) is also represented as Ar′SF₃.

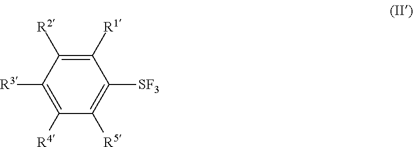

Similarly, when Ar═Ar′, the reaction of (I) (represented as ArSF₄X) and (IIIb) (represented as Ar′SR⁶) can give a single product (ArSF₃) as represented by the formula (II). When Ar≠Ar′, the reaction of (I) and (IIIb) provides a product of a mixture of (II) (ArSF₃) and (II′) (Ar′SF₃).

The R¹, R², R³, R⁴, and R⁵ of the products represented by the formula (II) may be different from the R¹, R², R³, R⁴, and R⁵ of the starting materials represented by the formula (I). And, the R^{1′}, R^{2′}, R^{3′}, R^{4′}, and R^{5′} of the products represented by the formula (II′) may be different from the R^{1′}, R^{2′}, R^{3′}, R^{4′}, and R^{5′} of the starting materials represented by the formulas (IIIa) and/or (IIIb). Thus, embodiments of this invention include transformation of the R¹, R², R³, R⁴, and R⁵ to another R¹, R², R³, R⁴, and R⁵ and of the R^{1′}, R^{2′}, R^{3′}, R^{4′}, and R^{5′} to another R^{1′}, R^{2′}, R^{3′}, R^{4′}, and R^{5′}, which may take place during the reaction of the present invention or under the reaction conditions as long as the —SF₄Cl and/or the —S—S— or —S— moiety is transformed to a —SF₃ group(s).

In order to get adequate production yields, the amount used of arylsulfur compound having a formula (IIIa) is around ⅙ moles per one mole of ArSF₄X. The amount used of arylsulfur compound having a formula (IIIb) is around ¼ moles per one mole of ArSF₄X when R₆≠a halogen atom. The amount used of arylsulfur compound having a formula (IIIb) is around ⅓ moles per one mole of ArSF₄X when R⁶═Cl, Br, or I. The amount used of arylsulfur compound having a formula (IIIb) is around ½ moles per one mole of ArSF₄X when R⁶═F. As above, other amounts can be used as long as the reaction proceeds.

The amount used of reducing substance other than the arylsulfur compound (IIIa) or (IIIb) can be selected for each reaction to obtain an adequate yield of arylsulfur trifluoride because the necessary amount of reducing substance is dependent on the kind and nature of the reducing substance and reaction conditions such as temperature and solvent in addition to the arylsulfur halotetrafluoride. An illustrative amount used of reducing substance per one mole of an arylsulfur halotetrafluoride is from about 0.1 moles to about ten moles and more typically about 0.1 moles to about five moles.

The reaction can be conducted in the absence or presence of solvent. In some cases, the reaction can preferably be conducted without a solvent. In other cases, solvent makes the reaction smooth, and for these cases, solvent is preferably selected from a group consisting of hydrocarbons, halocarbons, nitriles, aromatics, nitro compounds, esters, and mixture thereof. These solvents are exemplified as described above.

The reaction temperature primarily depends on the arylsulfur halotetrafluoride, reducing substance, and solvent used. The reaction temperature can be selected in the range of about −80° C. to about +200° C. When arylsulfur compounds having a formula (IIIa) or a formula (IIIb) (R⁶=a halogen atom) are used as a reducing substance, the reaction temperature is preferably selected from the range of about room temperature to about +150° C., and more preferably, about +50° C. to about +120° C. When arylsulfur compounds having a formula (IIIb) (R⁶≠a halogen atom) are used as a reducing substance, the reaction temperature can be preferably selected from the range of about −80° C. to about +150° C., and more preferably, about −50° C. to about +120° C. When inorganic or organic chloride salts are used as a reducing substance, the reaction temperature is preferably selected from the range of about +40° C. to about +150° C., more preferably, about +50° C. to about +120° C. When heteroaromatic compounds are used as a reducing substance, the reaction temperature is preferably selected in the range of about −50° C. to about +100° C., preferably, about −20° C. to about +70° C. When other reducing substances are used, the reaction temperature can be selected so that the reaction is finished in a reasonable time.

The reaction time is also dependent on reaction temperature, the arylsulfur halotetrafluorides, reducing substances, solvent, and their amounts used. Therefore, one can choose the time necessary for completing each reaction by modifying one or more these parameters, but can be from about 1 minute to several days, preferably, within a few days.

It will be understood by one of skill in the relevant art that certain compounds of the invention may comprise one or more chiral centers so that the compounds may exist as stereoisomers, including diastereoisomers and enantiomers. It is envisioned that all such compounds be within the scope of the present invention, including all such stereoisomers, and mixtures thereof, including racemates.

### EXAMPLES

The following examples are provided for illustrative purposes only and are not intended to limit the scope of the invention. Table 1 provides structure names and formulas for reference when reviewing the following examples:

TABLE 1
Substituted and Unsubstituted Phenylsulfur Halotetrafluorides (Formulas
IV~XIV):

| Formula Number | Name | Structure |
|---|---|---|
| IV | Phenylsulfur chlorotetrafluoride | 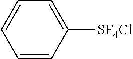 |
| V | p-Methylphenylsulfur chlorotetrafluoride | 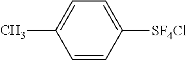 |
| VI | p-(tert-Butyl)phenylsulfur chlorotetrafluoride | 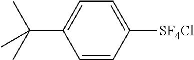 |
| VII | p-Fluorophenylsulfur chlorotetrafluoride | 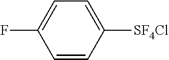 |
| VIII | o-Fluorophenylsulfur chlorotetrafluoride | 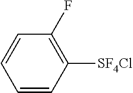 |
| IX | p-Chlorophenylsulfur chlorotetrafluoride | 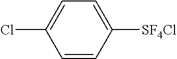 |
| X | p-Bromophenylsulfur chlorotetrafluoride | 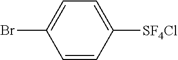 |
| XI | m-Bromophenylsulfur chlorotetrafluoride | 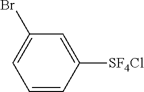 |
| XII | p-Nitrophenylsulfur chlorotetrafluoride | 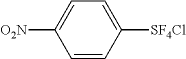 |
| XIII | 2,6-Difluorophenylsulfur chlorotetrafluoride | 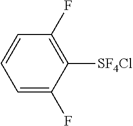 |
| XIV | 2,3,4,5,6- Pentafluorophenylsulfur chlorotetrafluoride | 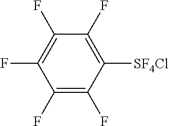 |

### Example 1

### Synthesis of Phenylsulfur Chlorotetrafluoride (IV) from Diphenyl Disulfide

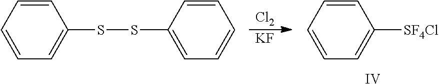

A 500 mL round bottom glassware flask was charged with diphenyl disulfide (33.0 g, 0.15 mol), dry KF (140 g, 2.4 mol) and 300 mL of dry CH₃CN. The stirred reaction mixture was cooled on an ice/water bath under a flow of N₂ (18 mL/min). After N₂ was stopped, chlorine (Cl₂) was bubbled into the reaction mixture at a rate of about 70 mL/min. The Cl₂ bubbling took about 6.5 h. The total amount of Cl₂ used was about 1.2 mol. After Cl₂ was stopped, the reaction mixture was stirred for additional 3 h. N₂ was then bubbled through for 2 hours to remove excess Cl₂. The reaction mixture was then filtered with 100 mL of dry hexanes in air. About 1 g of dry KF was added to the filtrate. The KF restrains possible decomposition of the product. The filtrate was evaporated under vacuum and the resulting residue was distilled at reduced pressure to give a colorless liquid (58.0 g, 88%) of phenylsulfur chlorotetrafluoride (formula IV): b.p. 80° C./20 mmHg; ¹H NMR (CD₃CN) δ 7.79-7.75 (m, 2H, aromatic), 7.53-7.49 (m, 3H, aromatic); ¹⁹F NMR (CD₃CN) δ 136.7 (s, SF₄Cl); High resolution mass spectrum; found 221.970281 (38.4%) (calcd for C₆H₅F₄S³⁷Cl; 221.970713), found 219.974359 (100%) (calcd for C₆H₅F₄S³⁵Cl; 219.973663). The NMR analysis showed phenylsulfur chlorotetrafluoride obtained as a trans isomer.

### Example 2

### Synthesis of Phenylsulfur Chlorotetrafluoride (IV) from Thiophenol

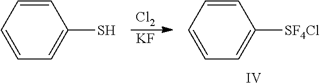

Chlorine (Cl₂) was passed with a flow rate of 27 mL/min into a stirred mixture of 10.0 g (90.8 mmol) of thiophenol and 47.5 g (0.817 mol) of dry KF in 100 mL of dry acetonitrile at 6˜10° C. Chlorine was passed for 3.7 h and the total amount of chlorine passed was 10.2 L (0.445 mol). The reaction mixture was filtered. After removal of the solvent in vacuum, phenylsulfur chlorotetrafluoride (IV) (16.6 g, 83%) as a light green-brown liquid was obtained. The physical properties and spectral data of the product are as shown in Example 1. The product was a trans isomer.

### Examples 3-12

### Synthesis of Arylsulfur Halotetrafluorides V˜XIV from Diaryl Disulfides or Arenethiols

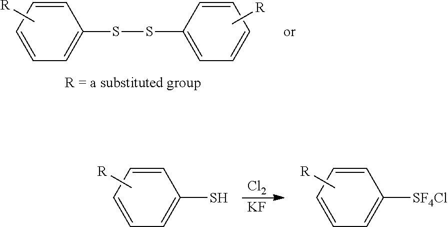

Substituted arylsulfur chlorotetrafluorides V˜XIV were synthesized from the corresponding diaryl disulfides or arenethiols by a similar procedure as shown in Example 1 or 2. Table 2 shows the synthesis of the substituted arylsulfur chlorotatrafluorides V˜XIV together with IV (Examples 1 and 2). Table 2 also shows the starting materials and other chemicals necessary for the synthesis, solvents, reaction conditions, and the results, together with those of Examples 1 and 2.

TABLE 2
Preparation of Arylsulfur Halotetrafluorides.

| | Disulfide or | | Fluoride | | | | | |
|---|---|---|---|---|---|---|---|---|
| Ex | Thiol | Halogen | source | Solvent | Temp* | Time* | ArSF₄X | Yield |
| 1 | 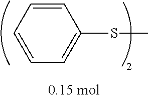 | Cl₂ ~1.2 mol | KF 2.4 mol | CH₃CN 300 mL | 0~5° C. | ~9.5 h | 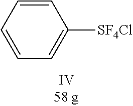 | 88% |
| 2 | 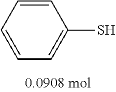 | Cl₂ 0.445 mol | KF 0.817 mol | CH₃CN 100 mL | 6~10° C. | 3.9 h | 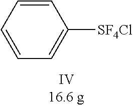 | 83% |
| 3 | 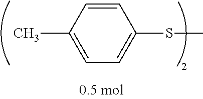 | Cl₂ 3.85 mol | KF 8 mol | CH₃CN 1000 mL | 0° C. | 10.5 h | 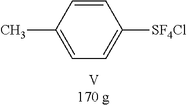 | 73% |
| 4 | 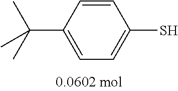 | Cl₂ 0.452 mol | CsF 0.602 mol | CH₃CN 150 mL | 5-10° C. and r.t. | ~5 h and ~24 h | 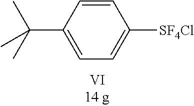 | 84% |
| 5 | 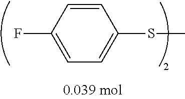 | Cl₂ 0.28 mol | KF 0.63 mol | CH₃CN 100 mL | 0~5° C. and r.t. | 2.5 h and o.n. | 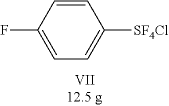 | 67% |
| 6 | 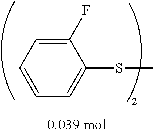 | Cl₂ 0.31 mol | KF 0.63 mol | CH₃CN 100 mL | 0~5° C. and r.t. | 1.8 h and o.n. | 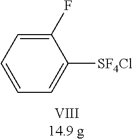 | 80% |
| 7 | 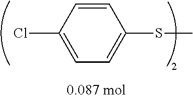 | Cl₂ 0.57 mol | KF 1.48 mol | CH₃CN 200 mL | 5~8° C. | 3.5 h | 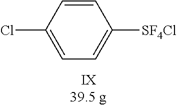 | 88% |
| 8 | 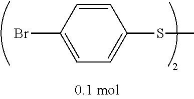 | Cl₂ 0.72 mol | KF 1.6 mol | CH₃CN 200 mL | 0~5° C. and r.t. | 4.5 h and o.n. | 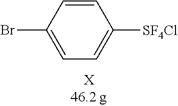 | 77% |
| 9 | 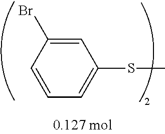 | Cl₂ 0.88 mol | KF 2.0 mol | CH₃CN 250 mL | 0~5° C. and r.t. | 5.5 h and o.n. | 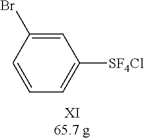 | 86% |
| 10 | 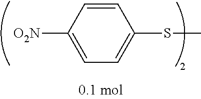 | Cl₂ 0.72 mol | KF 1.6 mol | CH₃CN 200 mL | 0~5° C. and r.t. | 4.5 h and o.n. | 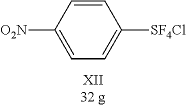 | 60% |
| 11 | 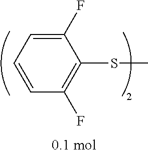 | Cl₂ 1.02 mol | CsF 1.83 mol | CH₃CN 200 mL | 0~5° C. and r.t. | 5 h and o.n. | 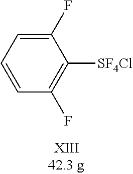 | 82% |
| 12 | 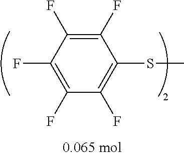 | Cl₂ ~1 mol | KF 1.41 mol | CH₃CN 300 mL | 0~5° C. and r.t. | 5 h and o.n. | 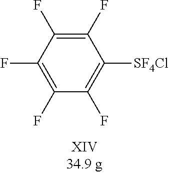 | 86% |

*r.t. = room temperature;
o.n. = overnight

The properties and spectral data of the products, (V)˜(XIV), obtained by Examples 3˜12 are shown by the following:

p-Methylphenylsulfur chlorotetrafluoride (V); b.p. 74-75° C./5 mmHg; ¹H NMR (CD₃CN) δ 7.65 (d, J=8.1 Hz, 2H, aromatic), 7.29 (d, J=8.1 Hz, 2H, aromatic), 2.36 (s, 3H, CH₃); ¹⁹F NMR (CD₃CN) δ 137.66 (s, SF₄Cl); High resolution mass spectrum; found 235.986234 (34.9%) (calcd for C₇H₇F₄S³⁷Cl; 235.986363), found 233.989763 (75.6%) (calcd for C₇H₇F₄S³⁵Cl; 233.989313). The NMR showed that p-methylphenylsulfur chlorotetrafluoride obtained was a trans isomer.

p-(tert-Butyl)phenylsulfur chlorotetrafluoride (VI); m.p. 93° C.; b.p. 98° C./0.3 mmHg; ¹H NMR (CDCl₃) δ 1.32 (s, 9H, C(CH₃)₃), 7.43 (d, J=9.2 Hz, 2H, aromatic), 7.64 (d, J=9.2 Hz, 2H, aromatic); ¹⁹F NMR δ138.3 (s, SF₄Cl). The NMR showed that p-(tert-butyl)phenylsulfur chlorotetrafluoride was obtained as a trans isomer. Elemental analysis; Calcd for C₁₀H₁₃ClF₄S; C, 43.40%; H, 4.74%. Found; C, 43.69%, H, 4.74%.

p-Fluorophenylsulfur chlorotetrafluoride (VII); b.p. 60° C./8 mmHg; ¹H NMR (CD₃CN) δ 7.85-7.78 (m, 2H, aromatic), 7.25-7.15 (m, 2H, aromatic); ¹⁹F NMR (CD₃CN) δ 137.6 (s, SF₄Cl), −108.3 (s, CF); High resolution mass spectrum; found 239.961355 (37.4%) (calcd for C₆H₄F₅S³⁷Cl; 239.961291), found 237.964201 (100%) (calcd for C₆H₄F₅S³⁵Cl; 237.964241). The NMR shows that p-fluorophenylsulfur chlorotetrafluoride was obtained as a trans isomer.

o-Fluorophenylsulfur chlorotetrafluoride (VIII); b.p. 96-97° C./20 mmHg; ¹H NMR (CD₃CN) δ 7.77-7.72 (m, 1H, aromatic), 7.60-7.40 (m, 1H, aromatic), 7.25-7.10 (m, 2H, aromatic); ¹⁹F NMR (CD₃CN) δ 140.9 (d, SF₄Cl), −107.6 (s, CF); High resolution mass spectrum; found 239.961474 (25.4%) (calcd for C₆H₄F₅S³⁷Cl; 239.961291), found 237.964375 (69.8%) (calcd for C₆H₄F₅S³⁵Cl; 237.964241). The NMR showed that o-fluorophenylsulfur chlorotetrafluoride was obtained as a trans isomer.

p-Chlorophenylsulfur chlorotetrafluoride (IX); b.p. 65-66° C./2 mmHg; ¹H NMR (CD₃CN) δ 7.65 (d, J=9.1 Hz, 2H, aromatic), 7.83 (d, J=9.1 Hz, 2H, aromatic); ¹⁹F NMR (CD₃CN) δ 137.4 (s, SF₄Cl); High resolution mass spectrum; found 257.927507 (13.3%) (calcd for C₆H₄F₄S³⁷Cl₂; 257.928790), found 255.930746 (68.9%) (calcd for C₆H₄F₄S³⁷Cl³⁵Cl; 255.931740), found 253.933767 (100.0%) (calcd for C₆H₄F₄S³⁵Cl₂; 253.934690). The NMR shows that p-chlorophenylsulfur chlorotetrafluoride was obtained as a trans isomer.

p-Bromophenylsulfur chlorotetrafluoride (X); m.p. 58-59° C.; ¹H NMR (CD₃CN) δ 7.67 (s, 4H, aromatic); ¹⁹F NMR (CD₃CN) δ 136.56 (s, SF₄Cl); High resolution mass spectrum; found 301.877066 (16.5%) (calcd for C₆H₄⁸¹Br³⁷ClF₄S; 301.879178), found 299.880655 (76.6%) (calcd for C₆H₄⁸¹Br³⁵ClF₄S; 299.881224 and calcd for C₆H₄⁷⁹Br³⁷ClF₄S; 299.882128), found 297.882761 (77.4%) (calcd for C₆H₄⁷⁹Br³⁵ClF₄S; 297.884174). The NMR showed that p-bromophenylsulfur chlorotetrafluoride was obtained as a trans isomer.

m-Bromophenylsulfur chlorotetrafluoride (XI); b.p. 57-59° C./0.8 mmHg; ¹H NMR (CD₃CN) δ 7.90-7.88 (m, 1H, aromatic), 7.70-7.50 (m, 2H, aromatic), 7.40-7.30 (m, 1H, aromatic); ¹⁹F NMR (CD₃CN) δ 136.74 (s, SF₄Cl); High resolution mass spectrum; found 301.878031 (29.1%) (calcd for C₆H₄⁸¹Br³⁷ClF₄S; 301.879178), found 299.881066 (100%) (calcd for C₆H₄⁸¹Br³⁵ClF₄S; 299.881224 and calcd for C₆H₄⁷⁹Br³⁷ClF₄S; 299.882128), found 297.883275 (77.4%) (calcd for C₆H₄⁷⁹Br³⁵ClF₄S; 297.884174). The NMR showed that m-bromophenylsulfur chlorotetrafluoride obtained was a trans isomer.

p-Nitrophenylsulfur chlorotetrafluoride (XII); m.p. 130-131° C.; ¹H NMR (CD₃CN) δ 8.29 (d, J=7.8 Hz, 2H, aromatic), 8.02 (d, J=7.8 Hz, 2H, aromatic); ¹⁹F NMR (CD₃CN) δ 134.96 (s, SF₄Cl); High resolution mass spectrum; found 266.956490 (38.4%) (calcd for C₆H₄³⁷ClF₄NO₂S; 266.955791), found 264.959223 (100%) (calcd for C₆H₄³⁵ClF₄NO₂S; 264.958741). The NMR showed that p-nitrophenylsulfur chlorotetrafluoride was obtained as a trans isomer.

2,6-Difluorophenylsulfur chlorotetrafluoride (XIII): The product (bp. 120-122° C./95-100 mmHg) obtained from Example 11 is a 6:1 mixture of trans- and cis-isomers of 2,6-difluorophenylsulfur chlorotetrafluoride. The trans-isomer was isolated as pure form by crystallization; mp. 47.6-48.3° C.; ¹⁹F NMR (CDCl₃) δ 143.9 (t, J=26.0 Hz, 4F, SF₄), −104.1 (quintet, J=26.0 Hz, 2F, Ar—F): ¹H NMR (CDCl₃) δ 6.97-7.09 (m, 2H, 3,5-H), 7.43-7.55 (m, 1H, 4-H); ¹³C NMR (CDCl₃) δ 157.20 (d, J=262.3 Hz), 133.74 (t, J=11.6 Hz), 130.60 (m), 113.46 (d, J=14.6 Hz); elemental analysis, C, 28.24%, H, 1.24% (calcd for C₆H₃ClF₆S; C, 28.08%, H, 1.18%); High resolution mass spectrum; found 257.950876 (37.6%) (calcd for C₆H₃³⁷ClF₆S; 257.951869), found 255.955740 (100%) (calcd for C₆H₃³⁵ClF₆S; 255.954819). The cis-isomer was assigned in the following; ¹⁹F NMR (CDCl₃) δ 158.2 (quartet, J=161.8 Hz, 1F, SF), 121.9 (m, 2F, SF₂), 76.0 (m, 1F, SF). The ¹⁹F NMR assignment of aromatic fluorine atoms of the cis-isomer could not be done because of possible overlapping of the peaks of the trans-isomer.

2,3,4,5,6-Pentafluorophenylsulfur chlorotetrafluoride (XIV): The product (b.p. 95-112° C./100 mmHg) obtained from Experiment 12 was a 1.7:1 mixture of trans and cis isomers of 2,3,4,5,6-pentafluorophenylsulfur chlorotetrafluoride. The isomers were assigned by ¹⁹F NMR: The trans isomer; ¹⁹F NMR (CDCl₃) δ 144.10 (t, J=26.0 Hz, 4F, SF₄), −132.7 (m, 2F, 2,6-F), −146.6 (m, 1F, 4-F), −158.9 (m, 2F, 3,5-F); ¹³C NMR (CDCl₃) δ 143.5 (dm, J=265.2 Hz), 141.7 (dm, J=263.7 Hz), 128.3 (m). The cis isomer; ¹⁹F NMR (CDCl₃) δ 152.39 (quartet, J=158.9 Hz, 1F, SF), 124.32 (m, 2F, SF₂), 79.4 (m, 1F, SF), −132.7 (m, 2F, 2,6-F), −146.6 (m, 1F, 4-F), −158.9 (m, 2F, 3,5-F). High resolution mass spectrum of a 1.7:1 mixture of the trans and cis isomers; found 311.923124 (15.5%) (calcd for C₆³⁷ClF₉S; 311.923604), found 309.926404 (43.1%) (calcd for C₆³⁵ClF₉S; 309.926554).

### Examples 13-29

### Fluorinations of Various Target Compounds with Arylsulfur Halotetrafluorides by a One-Step Process in Accordance with the Present Invention

A typical procedure in accordance with the embodiments of the present invention is as follows; a solution of 0.66 mmol of n-C₁₀H₂₁OC(S)SCH₃ in 1 mL of dry methylene chloride was added to a solution of 1.66 mmol of phenylsulfur chlorotetrafluoride in 3 mL of dry methylene chloride in a fluoropolymer (PFA) vessel under nitrogen atmosphere. The mixture was stirred at room temperature for 5 hours. ¹⁹F NMR analysis of the reaction mixture showed that n-C₁₀H₂₁OCF₃ was produced in a 96% yield.

Fluorinations of various target compounds with various arylsulfur halotetrafluorides were conducted in the same manner as above. Various fluorinated compounds were produced in high yields. Tables 3 shows the results and the detailed reaction conditions. The products were identified by comparison with known samples and/or spectral analyses.

TABLE 3
Fluorination of Various Target Compounds with ArSF₄Cl by a One-Step Process

| | | | | Temp | | | |
|---|---|---|---|---|---|---|---|
| | | | | & | Fluorinated | ¹⁹F-NMR | |
| Ex | ArSF₄Cl | Target Compound | Solvent | Time | Product | data | Yield |
| Ex | IV | n-C₁₀H₂₁OC(═S)SCH₃ | CH₂Cl₂ | r.t., | n-C₁₀H₂₁OCF₃ | −60.5 | 96% |
| 13 | (1.66 mmol) | (0.66 mmol) | (3 mL) | 5 h | | (s, CF₃) | |
| Ex | V | n-C₁₀H₂₁OC(═S)SCH₃ | CH₂Cl₂ | r.t., | n-C₁₀H₂₁OCF₃ | −60.5 | quant. |
| 14 | (3.21 mmol) | (2.14 mmol) | (3 mL) | 4 h | | (s, CF₃) | |
| Ex | VI | n-C₁₀H₂₁OC(═S)SCH₃ | CH₂Cl₂ | r.t., | n-C₁₀H₂₁OCF₃ | −60.5 | quant. |
| 15 | (1.48 mmol) | (0.59 mmol) | (2 mL) | 4 h | | (s, CF₃) | |
| Ex | VII | n-C₁₀H₂₁OC(═S)SCH₃ | CH₂Cl₂ | r.t., | n-C₁₀H₂₁OCF₃ | −60.5 | quant. |
| 16 | (3.18 mmol) | (2.13 mmol) | (mL) | 4 h | | (s, CF₃) | |
| Ex | VIII | n-C₁₀H₂₁OC(═S)SCH₃ | CH₂Cl₂ | r.t., | n-C₁₀H₂₁OCF₃ | −60.5 | 90% |
| 17 | (3.55 mmol) | (2.36 mmol) | (3 mL) | 4 h | | (s, CF₃) | |
| Ex | IX | n-C₁₀H₂₁OC(═S)SCH₃ | CH₂Cl₂ | r.t., | n-C₁₀H₂₁OCF₃ | −60.5 | quant. |
| 18 | (3.94 mmol) | (2.63 mmol) | (3 mL) | 4 h | | (s, CF₃) | |
| Ex | X | n-C₁₀H₂₁OC(═S)SCH₃ | CH₂Cl₂ | r.t., | n-C₁₀H₂₁OCF₃ | −60.5 | quant. |
| 19 | (2.73 mmol) | (1.82 mmol) | (3 mL) | 4 h | | (s, CF₃) | |
| Ex | XI | n-C₁₀H₂₁OC(═S)SCH₃ | CH₂Cl₂ | r.t., | n-C₁₀H₂₁OCF₃ | −60.5 | quant. |
| 20 | (3.37 mmol) | (2.24 mmol) | (3 mL) | 4 h | | (s, CF₃) | |
| Ex | XII | n-C₁₀H₂₁OC(═S)SCH₃ | CH₂Cl₂ | r.t., | n-C₁₀H₂₁OCF₃ | −60.5 | 94% |
| 21 | (5.50 mmol) | (3.66 mmol) | (3 mL) | 4 h | | (s, CF₃) | |
| Ex | XIII | n-C₁₀H₂₁OC(═S)SCH₃ | CH₂Cl₂ | r.t. | n-C₁₀H₂₁OCF₃ | −60.5 | 83% |
| 22 | (1.28 mmol) | (0.51 mmol) | (2 mL) | 24 h | | (s, CF₃) | |
| Ex | IV | PhC(═S)SCH₃ | CH₂Cl₂ | r.t., | PhCF₃ | −62.6 | 99% |
| 23 | (1.34 mmol) | (0.53 mmol) | (3 mL) | 40 h | | (s, CF₃) | |
| Ex | IV | PhC(═S)OCH₃ | neat | r.t., | PhCF₂OCH₃ | −72.2 | 60% |
| 24 | (4.5 mmol) | (6.74 mmol) | | 2 h | | (s, CF₃) | |
| Ex 25 | IV (3.29 mmol) | 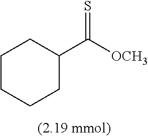 | CH₂Cl₂ (3 mL) | r.t., 5 h | 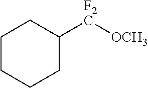 | −85.2 (s, CF₂) | quant. |
| Ex 26 | XIII (3.59 mmol) | 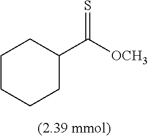 | CH₂Cl₂ (5 mL) | r.t., 4 h | 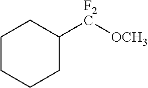 | −85.2 (s, CF₂) | 96% |
| Ex 27 | IV (0.92 mmol) | 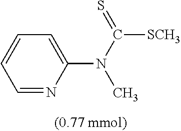 | CH₂Cl₂ (3 mL) | r.t., 48 h | 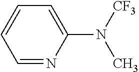 | −57.9 (s, CF₃) | 91% |
| Ex 28 | IV (2.02 mmol) | 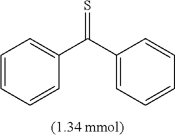 | CH₂Cl₂ (3 mL) | r.t., 5 h | 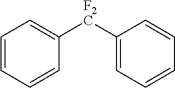 | −88.7 (s, CF₂) | quant. |
| Ex 29 | IV (1.86 mmol) | 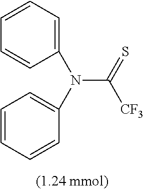 | CH₂Cl₂ (3 mL) | r.t., 3 h | 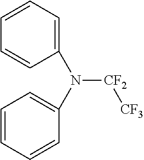 | −85.8 (s,CF₂), −79.8 (s,CF₃) | 75% |

Quant. = quantitative yield.

### Examples 30-41

### Fluorinations of Various Target Compounds with Arylsulfur Halotetrafluorides by a One-Step Process in the Presence of a Reducing Substance and in Accordance with the Present Invention

A typical procedure in accordance with embodiments of the present invention is as follows: 1 mmol of arylsulfur halotetrafluoride was added to a solution of 1 mmol benzoic acid and 1 mmol of pyridine in 2 mL of dry methylene chloride under nitrogen atmosphere in a fluoropolymer (PFA) vessel at room temperature. The reaction mixture was stirred for 1.5 hours at room temperature. ¹⁹F NMR analysis showed that benzoyl fluoride (PhCOF) was produced in a quantitative yield.

Fluorinations of target compounds with a different arylsulfur halotetrafluoride in the presence of a different reducing substance were conducted in the same manner as above. Table 4 shows results and detailed reaction conditions for other like embodiments. The products were identified by spectral analysis or by comparison with authentic samples. The yields of products were determined by the NMR analysis.

TABLE 4
Fluorination of Various Target Compounds with ArSF₄X by a One-Step-Process
in the Presence of a Different Reducing Substance.

| | | | | | | | | ¹⁹F | |
|---|---|---|---|---|---|---|---|---|---|
| | | Target | Reducing | | | | Fluorinated | NMR | |
| Ex | ASF₄X | Compound | Substance | Solvent | Temp | Time | Product | (ppm) | Yield |
| 30 | IV | PhCOOH | Pyridine | CH₂Cl₂ | r.t. | 1.5 h | PhCOF | 18.0 (s, | Quant. |
| | (1 mmol) | (1 mmol) | (1 mmol) | (2 mL) | | | | COF) | |
| 31 | IV | PhCOOH | (C₂H₅)₃N(HF)₃ | CH₂Cl₂ | r.t. | 1.5 h | PhCOF | 18.0 (s, | Quant. |
| | (1 mmol) | (1 mmol) | (1 mmol) | (2 mL) | | | | COF) | |
| 32 | IV | PhCOOH | Aniline | CH₂Cl₂ | r.t. | 5 h | PhCOF | 18.0 (s, | Quant. |
| | (1 mmol) | (1 mmol) | (1 mmol) | (2 mL) | | | | COF) | |
| 33 | IV | PhCOOH | Anthracene | CH₂Cl₂ | r.t. | 5 h | PhCOF | 18.0 (s, | Quant. |
| | (1 mmol) | (1 mmol) | (1 mmol) | (2 mL) | | | | COF) | |
| 34 | IV | PhCOOH | 2,3-dimethyl-2- | CH₂Cl₂ | r.t. | 1.5 h | PhCOF | 18.0 (s, | Quant. |
| | (1 mmol) | (1 mmol) | butene | (2 mL) | | | | COF) | |
| | | | (1 mmol) | | | | | | |
| 35 | IV | PhCOOH | Diphenyl | CH₂Cl₂ | r.t. | 1.5 h | PhCOF | 18.0 (s, | Quant. |
| | (1 mmol) | (1 mmol) | sulfide | (2 mL) | | | | COF) | |
| | | | (1 mmol) | | | | | | |
| 36 | IV | PhCOOH | Thiophenol | CH₂Cl₂ | r.t. | 1 h | PhCOF | 18.0 (s, | 91% |
| | (2 mmol) | (2 mmol) | (2 mmol) | (2 mL) | | | | COF) | |
| 37 | IV | PhCOOH | Mg (2.53 | THF | r.t. | 3 h | PhCOF | 18.0 (s, | 93% |
| | (2.53 | (2.53 mmol) | mmol), TBAI | (4 mL) | | | | COF) | |
| | mmol) | | (cat.) | | | | | | |
| 38 | IV | PhCOOH | Sn (1.75 mmol), | THF | r.t. | 2 h | PhCOF | 18.0 (s, | 84% |
| | (1.75 | (1.75 mmol) | TBAI (cat.) | (4 mL) | | | | COF) | |
| | mmol) | | | | | | | | |
| 39 | IV | PhCOOH | Zn (1.48 | THF | r.t. | 24 h | PhCOF | 18.0 (s, | 47% |
| | (1.48 | (1.48 mmol) | mmol), TBAI | (4 mL) | | | | COF) | |
| | mmol) | | (cat.) | | | | | | |
| 40 | XIII | PhCOOH | Pyridine | CH₂Cl₂ | r.t. | 1.5 h | PhCOF | 18.0 (s, | Quant. |
| | (1.01 | (1.01 mmol) | (1.01 mmol) | (2 mL) | | | | COF) | |
| | mmol) | | | | | | | | |
| 41 | XIV | PhCOOH | Pyridine | CH₂Cl₂ | r.t. | 1.5 h | PhCOF | 18.0 (s, | Quant. |
| | (1.48 | (1.48 mmol) | (1.48 mmol) | (3 mL) | | | | COF) | |
| | mmol) | | | | | | | | |
| 42 | IV (1.25 mmol) | 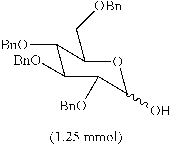 | Pyridine (1.25 mmol) | CH₂Cl₂ (2 mL) | r.t. | 5 h | 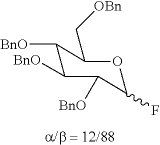 | −138.1 (β- isomer) −149.5 (α- isomer) | 83% |

Quant. = quantitative yield,
TBAI = tetrabutylammonioum iodide,
cat. = a catalytic amount,
THF = tetrahydrofuran.

### Examples 43-67

### Fluorinations of Various Target Compounds with Arylsulfur Halotetrafluorides by a Two-Step Process with a Reducing Substance and in Accordance with the Present Invention

A typical procedure for Ex. 43-51 in accordance with embodiments of the present invention is as follows; (step 1) 2.22 mmol of pyridine was added to a solution of 2.22 mmol of phenylsulfur chlorotetrafluoride in 2 mL of dry methylene chloride in a fluoropolymer (PFA) vessel at room temperature under nitrogen atmosphere. The reaction mixture was stirred for 1.5 hours at room temperature. At this point, ¹⁹F NMR analysis of the reaction mixture showed that phenylsulfur trifluoride was formed in high yield; and (step 2) 1.8 mmol of benzoic acid was added into the reaction mixture obtained by step 1, and the mixture was stirred at room temperature for 0.5 hours. ¹⁹F NMR showed that benzoyl fluoride was produced in a quantitative yield.

A typical procedure for Ex. 52-67 is as follows; (step 1) a solution of 0.65 mmol of diphenyl disulfide in 0.6 mL of dry methylene chloride was added dropwise to a stirred liquid of 3.95 mmol of phenylsulfur chlorotetrafluoride in a fluoropolymer vessel heated on an oil bath of 85° C. The methylene chloride (bp 40° C.) was removed from the reaction mixture just after the addition by means of vaporization on an oil bath heated at 85° C. After the addition of all the diphenyl disulfide, the reaction mixture was stirred for 0.5 h at 85° C. Chlorine (Cl₂) as a gaseous product evolved and was removed from the reaction mixture; and (step 2) 3 mL of dry methylene chloride and 2.63 mmol of n-dodecanol were added into the reaction mixture obtained by step 1, and the mixture was stirred at room temperature for 24 hours. ¹⁹F NMR showed that n-dodecyl fluoride was produced in 80% yield.

Fluorinations of various target compounds with various arylsulfur halotetrafluorides and various reducing substances were conducted in the same manner as above. Table 5 showed the results and the detailed reaction conditions. In Ex. 44-48, 52-67, in step 2, an additive and/or a solvent (shown in Table 5) was added into the reaction mixture in addition to a target compound. A small amount of ethanol added in step 2 of Ex. 54 and 55 reacted with arylsulfur trifluoride to form ethyl fluoride and hydrogen fluoride (HF), and the HF catalyzed the fluorination of the target compounds with arylsulfur trifluoride which remained. The products were identified by comparison with authentic samples and/or spectral analyses. The yields of products were determined by the NMR analysis.

The experimental examples of step 1 in Examples 43-67 are considered to be the experimental examples for the methods for preparing arylsulfur trifluorides in this invention.

TABLE 5
Fluorination of Various Target Compounds with ArSF₄X by a Two-Step Process with a Reducing Substance

| | Step 1 | | | | | Step 2 | | | | | | |
|---|---|---|---|---|---|---|---|---|---|---|---|---|
| | | | | | Product | | Addition | | | Fluorinated Product | | |
| | | Reducing | | Con- | of | | of | | Con- | | ¹⁹F | |
| Ex | ArSF₄X | substance | Solv. | ditions | Step 1 | Target compound | Solv. | Additives | ditions | Structure | NMR δ | Yield²⁾ |
| 43 | IV | Pyridine | CH₂Cl₂ | r.t., | PhSF₃ | PhCOOH | non | non | r.t. | PhCOF | 18.0 (s) | Quant. |
| | 2.22 mmol | 2.22 mmol | 2 mL | 1.5 h | | 1.8 mmol | | | 0.5 h | | | |
| 44 | IV | Pyridine | CH₂Cl₂ | r.t., | PhSF₃ | PhCHO | non | HF/py¹⁾ | r.t. | PhCF₂H | −110.5 | 87% |
| | 3.19 mmol | 3.19 mmol | 2 mL | 1.5 h | | 1.2 mmol | | 0.7 mL | 2 h | | (d, | |
| | | | | | | | | | | | J = 56 Hz) | |
| 45 | IV | Pyridine | CH₂Cl₂ | r.t., | PhSF₃ | n-C₁₂H₂₅OH | non | HF/py¹⁾ | r.t. | n-C₁₂H₂₅F | −217.9 | 86% |
| | 3.84 mmol | 3.84 mmol | 2 mL | 1.5 h | | 2.5 mmol | | 0.9 mL | 20 h | | (m) | |
| 46 | IV | Pyridine | CH₂Cl₂ | r.t., | PhSF₃ | PhCOCH₃ | non | HF/py¹⁾ | r.t. | PhCF₂CH₃ | −87.5 | 75% |
| | 3.30 mmol | 3.30 mmol | 2 mL | 1.5 h | | 1.32 mmol | | 0.8 mL | 20 h | | | |
| 47 | IV 3.5 mmol | Pyridine 3.5 mmol | CH₂Cl₂ 2 mL | r.t., 1.5 h | PhSF₃ | 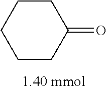 | non | HF/py¹⁾ 0.8 mL | r.t. 2 h | 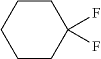 | −95.1 (br. s) | 94% |
| 48 | IV | Pyridine | CH₂Cl₂ | r.t., | PhSF₃ | n-C₁₀H₂₁COCH₃ | non | HF/py¹⁾ | r.t. | n-C₁₀H₂₁CF₂CH₃ | −90.2 | 86% |
| | 4.2 mmol | 4.2 mmol | 2 mL | 1.5 h | | 1.32 mmol | | 1 mL | 20 h | | | |
| 49 | IV | 2-Methoxy- | CH₂Cl₂ | −78° C. | PhSF₃ | PhCOOH | non | non | r.t. | PhCOF | 18.0 (s) | Quant. |
| | 1.68 mmol | 1-propene | 4 mL | to r.t., | 90%³⁾ | 1.2 mmol | | | 0.5 h | | | |
| | | 1.62 mmol | | 2 h to | | | | | | | | |
| | | | | 18 h | | | | | | | | |
| 50 | IV | (n-C₄H₉)₄NI | CH₃CN | r.t., | PhSF₃ | PhCOOH | non | non | r.t. | PhCOF | 18.0 (s) | Quant. |
| | 3.3 mmol | 1.65 mmol | 4 mL | 1 h | | 2.53 mmol | | | 1 h | | | |
| 51 | IV | KCl | CH₃CN | 80° C. | PhSF₃ | PhCOOH | non | non | r.t. | PhCOF | 18.0 (s) | Quant. |
| | 2 mmol | 2 mmol | 2 mL | 4 h | | 2 mmol | | | 1 h | | | |
| 52 | IV | Diphenyl | CH₂Cl₂ | 85° C. | PhSF₃ | n-C₁₂H₂₅OH | CH₂Cl₂ | non | r.t. | n-C₁₂H₂₅F | −217.9 | 80% |
| | 3.95 mmol | disulfide | 0.6 mL⁴⁾ | 0.5 h | | 2.63 mmol | 3 mL | | 24 h | | (m) | |
| | | 0.65 mmol | | | | | | | | | | |
| 53 | IV | Diphenyl | CH₂Cl₂ | 85° C. | PhSF₃ | n-C₁₀H₂₁CH(OH)CH₃ | CH₂Cl₂ | non | r.t. | n- | —171.9 | 71% |
| | 2.1 mmol | disulfide | 0.3 mL⁴⁾ | 0.5 h | | 1.39 mmol | 3 mL | | 24 h | C₁₀H₂₁CHFCH₃ | (m) | |
| | | 0.35 mmol | | | | | | | | | | |
| 54 | IV | Diphenyl | CH₂Cl₂ | 85° C. | PhSF₃ | PhCHO | CH₂Cl₂ | Ethanol | r.t. | PhCF₂H | —110.5 | 90% |
| | 2.72 mmol | disulfide | 0.4 mL⁴⁾ | 0.5 h | | 1.08 mmol | 2 mL | 40 μl | 2 h | | (d, | |
| | | 0.45 mmol | | | | | | | | | J = 56 Hz) | |
| 55 | IV 3.82 mmol | Diphenyl disulfide 0.63 mmol | CH₂Cl₂ 0.6 mL⁴⁾ | 85° C. 0.5 h | PhSF₃ | 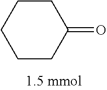 | CH₂Cl₂ 3 mL | Ethanol 50 μl | r.t. 24 h | 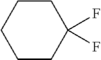 | —95.1 (br. s) | 80% |
| 56 | IV | Diphenyl | CH₂Cl₂ | 85° C. | PhSF₃ | PhCOOH | CH₂Cl₂ | non | r.t. | PhCOF | 18.0 (s) | Quant. |
| | 3.68 mmol | disulfide | 0.6 mL⁴⁾ | 0.5 h | | 1.22 mmol | 3 mL | | 2 h | | | |
| | | 0.61 mmol | | | | | | | | | | |
| 57 | IV | Diphenyl | CH₂Cl₂ | 85° C. | PhSF₃ | PhCOOH | non | HF/py¹⁾ | 50° C. | PhCF₃ | —62.6 (s) | 90% |
| | 2.63 mmol | disulfide | 0.4 mL⁴⁾ | 0.5 h | | 0.87 mmol | | 0.6 mL | 24 h | | | |
| | | 0.44 mmol | | | | | | | | | | |
| 58 | V | Diphenyl | CH₂Cl₂ | 85° C. | p- | PhCOOH | non | HF/py¹⁾ | 50° C. | PhCF₃ | —62.6 (m) | 77% |
| | 4.62 mmol | disulfide | 0.7 mL⁴⁾ | 0.5 h | CH₃C₆H₄SF₃ | 1.54 mmol | | 1.1 mL | 24 h | | | |
| | | 0.77 mmol | | | | | | | | | | |
| 59 | IX | Diphenyl | CH₂Cl₂ | 85° C. | p- | PhCOOH | non | HF/py¹⁾ | 50° C. | PhCF₃ | —62.6 (m) | 98% |
| | 4.95 mmol | disulfide | 0.8 mL⁴⁾ | 0.5 h | ClC₆H₄SF₃ | 1.63 mmol | | 1.3 mL | 24 h | | | |
| | | 0.83 mmol | | | | | | | | | | |
| 60 | IV | Diphenyl | CH₂Cl₂ | 85° C. | PhSF₃ | PhCOCl | non | HF/py¹⁾ | 50° C. | PhCF₃ | —62.6 (s) | 90% |
| | 4.65 mmol | disulfide | 0.7 mL⁴⁾ | 0.5 h | | 1.55 mmol | | 1.2 mL | 24 h | | | |
| | | 0.77 mmol | | | | | | | | | | |
| 61 | IV | Diphenyl | CH₂Cl₂ | 85° C. | PhSF₃ | p-(n- | non | HF/py¹⁾ | 50° C. | p-(n- | —62.1 (s) | Quant. |
| | 4.99 mmol | disulfide | 0.8 mL⁴⁾ | 0.5 h | | C₇H₁₅)C₆H₄COOH | | 1.2 mL | 24 h | C₇H₁₅)C₆H₄CF₃ | | |
| | | 0.83 mmol | | | | 1.66 mmol | | | | | | |
| 62 | IV | Diphenyl | CH₂Cl₂ | 85° C. | PhSF₃ | n-C₁₁H₂₃COOH | non | HF/py¹⁾ | 50° C. | n-C₁₁H₂₃CF₃ | —66.4 (s) | 96% |
| | 3.94 mmol | disulfide | 0.6 mL⁴⁾ | 0.5 h | | 1.30 mmol | | 0.9 mL | 24 h | | | |
| | | 0.66 mmol | | | | | | | | | | |
| 63 | IV 3.38 mmol | Diphenyl disulfide 0.56 mmol | CH₂Cl₂ 0.5 mL⁴⁾ | 85° C. 0.5 h | PhSF₃ | 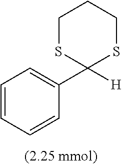 | CH₂Cl₂ 4 mL | non | r.t. 2 h | PhCF₂H | —110.5 (d, J = 56 Hz) | 99% |
| 64 | IV | Diphenyl | CH₂Cl₂ | 85° C. | PhSF₃ | PhC(═S)Ph | CH₂Cl₂ | non | r.t. | PhCF₂Ph | —88.7 (s) | 94% |
| | 4.72 mmol | disulfide | 0.7 mL⁴⁾ | 0.5 h | | 3.14 mmol | 4 mL | | 2 h | | | |
| | | 0.78 mmol | | | | | | | | | | |
| 65 | IV | Diphenyl | CH₂Cl₂ | 85° C. | PhSF₃ | PhC(═S)OCH₃ | CH₂Cl₂ | non | r.t. | PhCF₂OCH₃ | —72.2 (s) | 98% |
| | 2.43 mmol | disulfide | 0.5 mL⁴⁾ | 0.5 h | | 0.97 mmol | 2 mL | | 3 h | | | |
| | | 0.405 mmol | | | | | | | | | | |
| 66 | IV | Diphenyl | CH₂Cl₂ | 85° C. | PhSF₃ | n- | CH₂Cl₂ | non | r.t. | n-C₁₀H₂₁OCF₃ | —60.5 (s) | 90% |
| | 4.35 mmol | disulfide | 0.7 mL⁴⁾ | 0.5 h | | C₁₀H₂₁OC(═S)SCH₃ | 4 mL | | 3 h | | | |
| | | 0.72 mmol | | | | 1.45 mmol | | | | | | |
| 67 | IV 4.31 mmol | Diphenyl disulfide 0.72 mmol | CH₂Cl₂ 0.7 mL⁴⁾ | 85° C. 0.5 h | PhSF₃ | 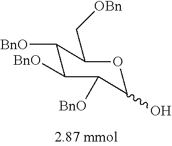 | CH₂Cl₂ 3 mL | non | r.t. 3 h | 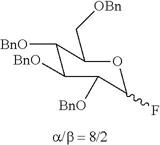 | α- isomer; 149 (dd) β- isomer; 138 d) | Quant. |

¹⁾HF/py; a mixture of HF and pyridine (HF:pyridine = about 70 wt %:about 30 wt %) was used.
²⁾Quant = a quantitative yield.
³⁾The ¹⁹F NMR analysis using a standard compound showed that the product, phenylsulfur trifluoride, of the step 1 was produced in a 90% yield.
⁴⁾This solvent was used to make a solution of diphenyl disulfide for the dropwise addition (see the procedure as described for Ex. 52~67 in the text above).

### Examples 68

### Preparation of Phenylsulfur Trifluorides from Phenylsulfur Chlorotetrafluorides with Pyridine (as a Reducing Substance)

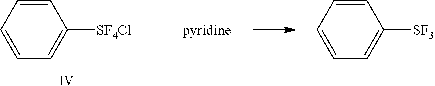

Under nitrogen atmosphere, pyridine (0.79 g, 10 mmol) was added to a solution of phenylsulfur chlorotetrafluoride (2.205 g, 10 mmol) in 5 mL of dry methylene chloride in a fluoropolymer (PFA) vessel at room temperature. The reaction mixture was stirred at room temperature for 1.5 hours. After the reaction, the reaction solvent was removed under vacuum and the residue was distilled under reduced pressure to give 1.46 g (88%) of phenylsulfur trifluoride (bp. 70° C./10 mmHg). ¹⁹F NMR (CD₃CN) δ 57.84 (br.s, 2F), −41.99 (br.s, 1F).

### Examples 69

### Preparation of 2,6-Difluorophenylsulfur Trifluoride from 2,6-Difluorophenylsulfur Chlorotetrafluoride with Pyridine (as a Reducing Substance)

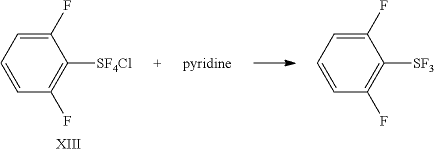

Under nitrogen atmosphere, pyridine (75 mg, 0.93 mmol) was added to a solution of 2,6-difluorophenylsulfur chlorotetrafluoride (a 6:1 mixture of trans- and cis-isomers) (240 mg, 0.93 mmol) in 2 mL of dry methylene chloride in a fluoropolymer (PFA) vessel at room temperature. The reaction mixture was stirred at room temperature for 1.5 hours. NMR analysis of the reaction mixture showed that 2,6-difluorophenylsulfur trifluoride was produced in 99% yield. ¹⁹F NMR (CD₃CN) δ 65.85 (dt, J=72.9, 11.2 Hz, SF₂), −55.22 (m, SF), −110.6 (m, aromatic F), −112.9 (m, aromatic F).

### Examples 70

### Preparation of Phenylsulfur Trifluoride from Phenylsulfur Chlorotetrafluoride with KCl (as a Reducing Substance)

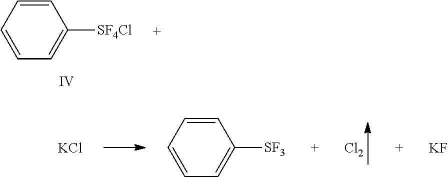

Phenylsulfur chlorotetrafluoride (3.5 g, 15.87 mmol), dry acetonitrile (7 mL), and potassium chloride (KCl, 3.5 g, 47 mmol) were put in a fluoropolymer (PFA) reactor equipped with a magnetic stirrer, a condenser, and a gas exit. The mixture was heated at 85° C. for 5 h on an oil bath. During that time, gas (chlorine, Cl₂) was evolved, which was detected with a paper soaked with an aqueous KI solution. After the reaction, the reaction mixture was cooled to room temperature and filtered under nitrogen atmosphere. Acetonitrile was removed at reduced pressure and the residue was distilled to give phenylsulfur trifluoride (2.2 g, by 70-71° C./10 mmHg, 85% yield). NMR data are shown in Example 65.

### Examples 71

### Preparation of Phenylsulfur Trifluorides from Phenylsulfur Chlorotetrafluoride with Diphenyl Disulfide (as a Reducing Substance)

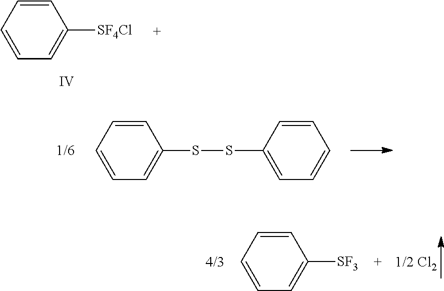

Phenylsulfur chlorotetrafluoride (2.18 g, 9.87 mmol) was put in a fluoropolymer (PFA) reactor equipped with a magnetic stirrer, a condenser, and a gas exit. The reactor was heated to 85° C. on an oil bath, and a solution of 0.359 g (1.64 mmol) of diphenyl disulfide in 1 mL of dry methylene chloride was added dropwise for 10 min. Evolution of gas (Cl₂) was started after about 15 min. Heating at 85° C. was continued till the evolution of chlorine stopped. It took 0.75 h. After the reaction, the reaction mixture was distilled under reduced pressure to give 1.96 g (11.8 mmol) (90% yield) of phenylsulfur trifluoride (bp. 70° C./10 mmHg). NMR data are shown in Example 65. The molar amount of the obtained product (phenylsulfur trifluoride) was 1.2 times molar amount of the molar amount of the starting material (phenylsulfur chlorotetrafluoride) used.

The gas (Cl₂) generated from the reaction was passed through a solution of trans-stilbene (1.44 g, 8 mmol) in 10 mL of methylene chloride at ice water temperature. After the reaction, the reaction solution was evaporated up to dryness to give the solid (1.72 g). ¹H NMR and GC-Mass analyses of the solid showed that an about 1.5:1 mixture of two isomeric 1,2-dichlorostilbene was produced. The GC-Mass spectral data agreed with the authentic sample. The weight increase of the product (1.72 g) from stilbene (1.44 g) used was 280 mg (3.94 mmol as Cl₂) which corresponded to the amount of chlorine gas generated. The amount of Cl₂ generated was calculated to be at least 80% yield on the basis of the theoretical amount (4.94 mmol). This experiment demonstrated that chlorine (Cl₂) was generated from the reaction of PhSF₄Cl and diphenyl disulfide.

### Examples 72

### Preparation of p-Chlorophenylsulfur Trifluorides from p-Chlorophenylsulfur Chlorotetrafluorides with Bis(p-Chlorophenyl) Disulfide (as a Reducing Substance)

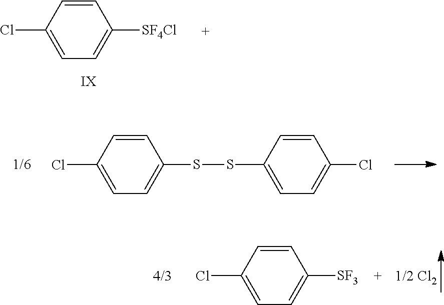

p-Chlorophenylsulfur chlorotetrachloride (2.55 g, 10 mmol) was put in a fluoropolymer (PFA) vessel equipped with a magnetic stirrer, a condenser made of fluoropolymer (PFA), and a gas exit. A solution of bis(p-chlorophenyl) disulfide (0.477 g, 1.67 mmol) in 0.5 mL of dry methylene chloride was added in portion wise to the fluoropolymer vessel heated at 85° C. for 10 min. After about 20 min, evolution of gas (Cl₂) was started, which was checked by a paper soaked with an aqueous KI solution. Heating was continued till the evolution of Cl₂ ceased. It took about 2.25 hours. After that, the reaction mixture was cooled to room temperature and distilled under reduced pressure to give 2.38 g (11.9 mmol) (89% yield) of p-chlorophenylsulfur trifluoride; bp. 56° C./1 mmHg. ¹⁹F NMR (CD₃CN) δ 55.59 (br.s, 2F), −40.60 (br.s, 1F).

The molar amount of the obtained product (p-chlorophenylsulfur trifluoride) was 1.2 times molar amount of the starting material (p-chlorophenylsulfur chlorotetrafluoride) used.

### Examples 73

### Preparation of Phenylsulfur Trifluoride and Chlorophenylsulfur Trifluoride from Phenylsulfur Chlorotetrafluorides with Thiophenol (as a Reducing Substance)

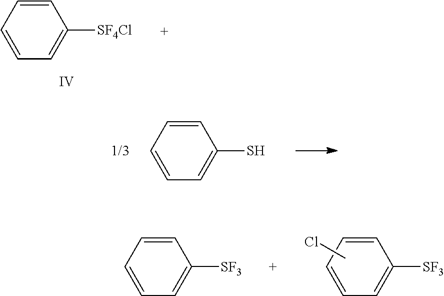

Phenylsulfur chlorotetrafluoride (2.732 g, 12.39 mmol) was put in a fluoropolymer (PFA) reactor equipped with a magnetic stirrer, a condenser, and a gas exit. The reactor was heated to 85° C. on an oil bath, and a solution of 0.452 g (4.10 mmol) of thiophenol in 0.5 mL of dry methylene chloride was added dropwise for 10 min. Evolution of gas started immediately. The gas oxidized an aqueous KI solution. The gas was assumed to be a mixture of chlorine (Cl₂), hydrogen fluoride, and hydrogen chloride. Heating at 85° C. was continued till the evolution of gas stopped. It took about 0.5 h. After the reaction, the reaction mixture was distilled under reduced pressure to give 2.4 g of a liquid (bp 70-71° C./10 mmHg), which was found that a 2:1 mixture of phenylsulfur trifluoride and chlorophenylsulfur trifluoride was produced in a total 81% yield, by NMR and GC-Mass analyses. ¹⁹F-NMR (CD₃CN) of phenylsulfur trifluoride; δ 57.94 (d, J=58 Hz, SF₂), −41.73 (t, J=58 Hz, SF). ¹⁹F-NMR (CD₃CN) of chlorophenylsulfur trifluoride; δ 57.75 (d, J=58 Hz, SF₂), −40.13 (t, J=58 Hz, SF). The position of the chlorine atom on the benzene ring was not determined. For the GC-Mass measurement, the product was treated with methanol and the resulting reaction mixture was measured by GC-Mass. The GC-Mass detected methyl phenylsulfinate and methyl chlorophenylsulfinate, which were derived from phenylsulfur trifluoride and chlorophenylsulfur trifluoride by the reaction with methanol, respectively.

The total molar amount (12.8 mmol) of the obtained products (phenylsulfur trifluoride and p-chlorophenylsulfur trifluoride) was 1.03 times molar amount of the starting material (phenylsulfur chlorotetrafluoride) used. Both phenylsulfur trifluoride and chlorophenylsulfur trifluoride are fluorinating agents.

### Examples 74

### Preparation of p-(Tert-Butyl)Phenylsulfur Trifluorides from p-(Tert-Butyl)Phenylsulfur Chlorotetrafluorides with Bis[p-(Tert-Butyl)Phenyl] Disulfide (as a Reducing substance)

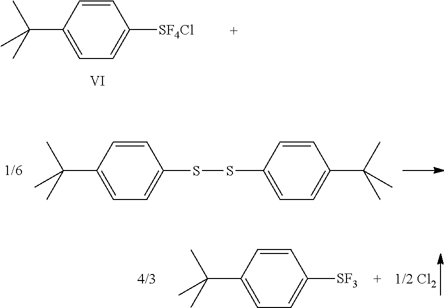

p-(tert-Butyl)phenylsulfur chlorotetrachloride (2.765 g, 10 mmol) was put in a fluoropolymer (PFA) vessel equipped with a magnetic stirrer, a condenser made of fluoropolymer (PFA), and a gas exit. A solution of bis[p-(tert-butyl)phenyl] disulfide (0.548 g, 1.66 mmol) in 0.5 mL of dry methylene chloride was added in portion wise to the fluoropolymer vessel heated at 95° C. for 10 min. After about 15 min, evolution of gas (Cl₂) was started, which was checked by a paper soaked with an aqueous KI solution. Heating was continued till the evolution of Cl₂ ceased. It took about 0.75 hours. After that, the reaction mixture was cooled to room temperature and distilled under reduced pressure to give 2.71 g (12.2 mmol) (92% yield) of p-(tert-butyl)phenylsulfur trifluoride; bp. 76° C./1 mmHg. ¹H-NMR (CDCl₃) δ 1.36 (s, 9H), 7.58 (d, J=9 Hz, 2H), 7.95 (d, J=9 Hz, 2H). ¹⁹F NMR (CDCl₃-Et₂O) δ 55.91 (d, J=54.5 Hz, 2F), −37.01 (t, J=54.5 Hz, 1F). The molar amount of the obtained product [p-(tert-butyl)phenylsulfur trifluoride] was 1.22 times molar amount of the starting material [p-(tert-butyl)phenylsulfur chlorotetrafluoride] used.

It is understood for purposes of this disclosure, that various changes and modifications may be made to the invention that are well within the scope of the invention. Numerous other changes may be made which will readily suggest themselves to those skilled in the art which are encompassed in the spirit of the invention disclosed herein and as defined in the appended claims.

This specification contains numerous citations to references such as patents, patent applications, and publications. Each is hereby incorporated by reference for all purposes.

What is claimed is:

1. A process for introducing one or more fluorine atoms into a target compound with arylsulfur halotetrafluoride as represented by formula (I): 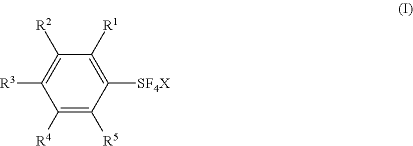
wherein X is a chlorine atom, bromine atom, or iodine atom, and R¹, R², R³, R⁴, and R⁵ each is independently a hydrogen atom, a halogen atom, a substituted or unsubstituted linear, branched, or cyclic alkyl group having one to ten carbon atoms, a nitro group, a cyano group, a substituted or unsubstituted aryl group having six to sixteen carbon atoms, a substituted or unsubstituted alkanesulfonyl group having one to ten carbon atoms, a substituted or unsubstituted arenesulfonyl group having six to sixteen carbon atoms, a substituted or unsubstituted alkoxy group having one to ten carbon atoms, a substituted or unsubstituted aryloxy group having six to sixteen carbon atoms, or a SF₅ group.

2. The process of claim 1, wherein X is a chlorine atom.

3. The process of claim 1, wherein R¹, R², R³, R⁴, and R⁵ each is independently selected from a group consisting of a hydrogen atom, a halogen atom, a substituted or unsubstituted linear or branched alkyl group having one to four carbon atoms, and a nitro group.

4. The process of claim 1, wherein all of R¹, R², R³, R⁴, and R⁵ are a hydrogen atom, or at maximum three of R¹, R², R³, R⁴, and R⁵ each is independently selected from a group consisting of a halogen atom, a substituted or unsubstituted linear or branched alkyl group having one to four carbon atoms, and a nitro group, and the remainders are a hydrogen atom.

5. The process of claim 1, wherein the arylsulfur halotetrafluoride is selected from a group consisting of phenylsulfur chlorotetrafluoride, o-, m-, and p-alkylphenylsulfur chlorotetrafluorides wherein the alkyl is a linear or branched alkyl group having one to four carbon atoms, o-, m-, and p-fluorophenylsulfur chlorotetrafluorides, o-, m-, and p-chlorophenylsulfur chlorotetrafluorides, o-, m-, and p-bromophenylsulfur chlorotetrafluorides, o-, m-, and p-nitrophenylsulfur chlorotetrafluorides, and each isomer of difluorophenylsulfur chlorotetrafluoride.

6. The process of claim 1, comprising contacting the target compound with arylsulfur halotetrafluoride as represented by the formula (I).

7. The process of claim 6, wherein X is a chlorine atom.

8. The process of claim 6, wherein R¹, R², R³, R⁴, and R⁵ each is independently selected from a group consisting of a hydrogen atom, a halogen atom, a substituted or unsubstituted linear or branched alkyl group having one to four carbon atoms, and a nitro group.

9. The process of claim 6, wherein all of R¹, R², R³, R⁴, and R⁵ are a hydrogen atom, or at maximum three of R¹, R², R³, R⁴, and R⁵ each is independently selected from a group consisting of a halogen atom, a substituted or unsubstituted linear or branched alkyl group having one to four carbon atoms, and a nitro group, and the remainders are a hydrogen atom.

10. The process of claim 6, wherein the arylsulfur halotetrafluoride is selected from a group consisting of phenylsulfur chlorotetrafluoride, o-, m-, and p-alkylphenylsulfur chlorotetrafluorides wherein the alkyl is a linear or branched alkyl group having one to four carbon atoms, o-, m-, and p-fluorophenylsulfur chlorotetrafluorides, o-, m-, and p-chlorophenylsulfur chlorotetrafluorides, o-, m-, and p-bromophenylsulfur chlorotetrafluorides, o-, m-, p-nitrophenylsulfur chlorotetrafluorides, and each isomer of difluorophenylsulfur chlorotetrafluoride.

11. The process of claim 1, comprising contacting the target compound with the arylsulfur halotetrafluoride represented by the formula (I) in the presence of a reducing substance that reduces the arylsulfur halotetrafluoride.

12. The process of claim 11, wherein X of the halotetrafluoride is a chlorine atom.

13. The process of claim 11, wherein the arylsulfur halotetrafluoride in which R¹, R², R³, R⁴, and R⁵ each is independently selected from a group consisting of a hydrogen atom, a halogen atom, a substituted or unsubstituted linear or branched alkyl group having one to four carbon atoms, and a nitro group.

14. The process of claim 11, wherein all of R¹, R², R³, R⁴, and R⁵ are a hydrogen atom, or at maximum three of R¹, R², R³, R⁴, and R⁵ each is independently selected from a group consisting of a halogen atom, a substituted or unsubstituted linear or branched alkyl group having one to four carbon atoms, and a nitro group, and the remainders are a hydrogen atom.

15. The process of claim 11, wherein the arylsulfur halotetrafluoride is selected from a group consisting of phenylsulfur chlorotetrafluoride, o-, m-, and p-alkylphenylsulfur chlorotetrafluorides wherein the alkyl is a linear or branched alkyl group having one to four carbon atoms, o-, m-, and p-fluorophenylsulfur chlorotetrafluorides, o-, m-, and p-chlorophenylsulfur chlorotetrafluorides, o-, m-, and p-bromophenylsulfur chlorotetrafluorides, o-, m-, and p-nitrophenylsulfur chlorotetrafluorides, and each isomer of difluorophenylsulfur chlorotetrafluoride.

16. The process of claim 11, wherein the reducing substance is a substance which has reduction potential that is lower than that of arylsulfur halotetrafluoride as represented by formula (I) used in the reaction.

17. The process of claim 11, wherein the reducing substance is at least one substance selected from a group consisting of elements and inorganic and organic compounds that reduce arylsulfur halotetrafluoride as represented by formula (I) used in the reaction.

18. The process of claim 17, wherein the elements are alkali metals, alkali earth metals, transition metals, metals in Groups 13˜15 of the Periodic Table, and semi-metals; the inorganic compounds are inorganic chloride salts, inorganic bromide salts, inorganic iodide salts; and the organic compounds are organic chloride salts, organic bromide salts, organic iodide salts, substituted and unsubstituted aromatic hydrocarbons, substituted and unsubstituted heteroaromatic compounds, substituted and unsubstituted unsaturated aliphatic hydrocarbons, substituted and unsubstituted nitrogen-containing aliphatic hydrocarbons, organic sulfur compounds, salts or complexes of substituted or unsubstituted heteroaromatic compounds and hydrogen fluoride, and salts or complexes of substituted or unsubstituted nitrogen-containing aliphatic hydrocarbons and hydrogen fluoride.

19. The process of claim 11, wherein the reducing substance is at least one substance selected from a group consisting of elements and inorganic and organic compounds that reduce the arylsulfur halotetrafluoride represented by the formula (I) to arylsulfur trifluoride represented by the formula (II): 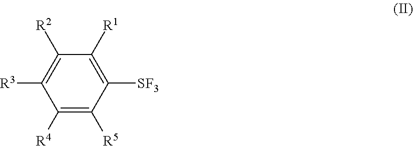
wherein R¹, R², R³, R⁴, and R⁵ each is independently a hydrogen atom, a halogen atom, a substituted or unsubstituted linear, branched, or cyclic alkyl group having one to ten carbon atoms, a nitro group, a cyano group, a substituted or unsubstituted aryl group having six to sixteen carbon atoms, a substituted or unsubstituted alkanesulfonyl group having one to ten carbon atoms, a substituted or unsubstituted arenesulfonyl group having six to sixteen carbon atoms, a substituted or unsubstituted alkoxy group having one to ten carbon atoms, a substituted or unsubstituted aryloxy group having six to sixteen carbon atoms, or a SF₅ group.

20. The process of claim 19, wherein the elements are alkali metals, alkali earth metals, transition metals, metals in Groups 13˜15 of the Periodic Table, and semi-metals; the inorganic compounds are inorganic chloride salts, inorganic bromide salts, and inorganic iodide salts; and the organic compounds are organic chloride salts, organic bromide salts, organic iodide salts, substituted and unsubstituted aromatic hydrocarbons, substituted and unsubstituted heteroaromatic compounds, substituted and unsubstituted unsaturated aliphatic hydrocarbons, substituted and unsubstituted nitrogen-containing aliphatic hydrocarbons, organic sulfur compounds, salts or complexes of substituted or unsubstituted heteroaromatic compounds and hydrogen fluoride, and salts or complexes of substituted or unsubstituted nitrogen-containing aliphatic hydrocarbons and hydrogen fluoride.

21. The process of claim 1, comprising; (step 1) contacting arylsulfur halotetrafluoride represented by the formula (I) with a reducing substance that reduces the arylsulfur halotetrafluoride, and then (step 2) contacting the target compound with the resulting mixture from step 1.

22. The process of claim 21, wherein X of the arylsulfur halotetrafluoride is a chlorine atom.

23. The process of claim 21, wherein the arylsulfur halotetrafluoride in which R¹, R², R³, R⁴, and R⁵ each is independently selected from a group consisting of a hydrogen atom, a halogen atom, a substituted or unsubstituted linear or branched alkyl group having one to four carbon atoms, and a nitro group.

24. The process of claim 21, wherein all of R¹, R², R³, R⁴, and R⁵ are a hydrogen atom, or at maximum three of R¹, R², R³, R⁴, and R⁵ each is independently selected from a group consisting of a halogen atom, a substituted or unsubstituted linear or branched alkyl group having one to four carbon atoms, and a nitro group, and the remainders are a hydrogen atom.

25. The process of claim 21, wherein the arylsulfur halotetrafluoride is selected from a group consisting of phenylsulfur chlorotetrafluoride, o-, m-, and p-alkylphenylsulfur chlorotetrafluorides wherein the alkyl is a linear or branched alkyl group having one to four carbon atoms, o-, m-, and p-fluorophenylsulfur chlorotetrafluorides, o-, m-, and p-chlorophenylsulfur chlorotetrafluorides, o-, m-, and p-bromophenylsulfur chlorotetrafluorides, o-, m-, and p-nitrophenylsulfur chlorotetrafluorides, and each isomer of difluorophenylsulfur chlorotetrafluoride.

26. The process of claim 21, wherein the reducing substance is a substance which has reduction potential that is lower than that of arylsulfur halotetrafluoride represented by the formula (I) used in the reaction.

27. The process of claim 21, wherein the reducing substance is at least one substance selected from a group consisting of elements and inorganic and organic compounds that reduce arylsulfur halotetrafluoride represented by the formula (I) used in the reaction.

28. The process of claim 27, wherein the elements are alkali metals, alkali earth metals, transition metals, metals in Groups 13˜15 of the Periodic Table, and semi-metals; the inorganic compounds are inorganic chloride salts, inorganic bromide salts, and inorganic iodide salts; and the organic compounds are organic chloride salts, organic bromide salts, organic iodide salts, substituted and unsubstituted aromatic hydrocarbons, substituted and unsubstituted heteroaromatic compounds, substituted and unsubstituted unsaturated aliphatic hydrocarbons, substituted and unsubstituted nitrogen-containing aliphatic hydrocarbons, organic sulfur compounds, salts or complexes of substituted or unsubstituted heteroaromatic compounds and hydrogen fluoride, and salts or complexes of substituted or unsubstituted nitrogen-containing aliphatic hydrocarbons and hydrogen fluoride.

29. The process of claim 21, wherein the reducing substance is at least one substance selected from a group consisting of elements and inorganic and organic compounds that reduce the arylsulfur halotetrafluoride represented by the formula (I) to arylsulfur trifluoride represented by the formula (II): 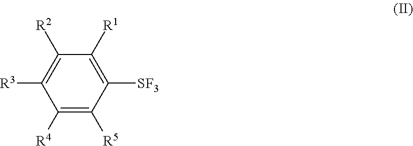
wherein R¹, R², R³, R⁴, and R⁵ each is independently a hydrogen atom, a halogen atom, a substituted or unsubstituted linear, branched, or cyclic alkyl group having one to ten carbon atoms, a nitro group, a cyano group, a substituted or unsubstituted aryl group having six to sixteen carbon atoms, a substituted or unsubstituted alkanesulfonyl group having one to ten carbon atoms, a substituted or unsubstituted arenesulfonyl group having six to sixteen carbon atoms, a substituted or unsubstituted alkoxy group having one to ten carbon atoms, a substituted or unsubstituted aryloxy group having six to sixteen carbon atoms, or a SF₅ group.

30. The process of claim 29, the elements are alkali metals, alkali earth metals, transition metals, metals in Groups 13˜15 of the Periodic Table, and semi-metals; the inorganic compounds are inorganic chloride salts, inorganic bromide salts, and inorganic iodide salts; and the organic compounds are organic chloride salts, organic bromide salts, organic iodide salts, substituted and unsubstituted aromatic hydrocarbons, substituted and unsubstituted heteroaromatic compounds, substituted and unsubstituted unsaturated aliphatic hydrocarbons, substituted and unsubstituted nitrogen-containing aliphatic hydrocarbons, organic sulfur compounds, salts or complexes of substituted or unsubstituted heteroaromatic compounds and hydrogen fluoride, and salts or complexes of substituted or unsubstituted nitrogen-containing aliphatic hydrocarbons and hydrogen fluoride.

31. The process of claim 1, comprising; (step 1) contacting arylsulfur halotetrafluoride represented by the formula (I) with a reducing substance to form arylsulfur trifluoride represented by the formula (II), and then (step 2) contacting the target compound with the arylsulfur trifluoride obtained from step 1; wherein formula (II) is represented by: 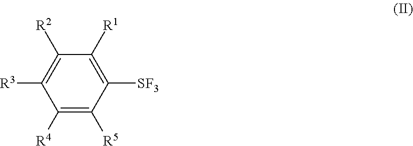
wherein R¹, R², R³, R⁴, and R⁵ each is independently a hydrogen atom, a halogen atom, a substituted or unsubstituted linear, branched, or cyclic alkyl group having one to ten carbon atoms, a nitro group, a cyano group, a substituted or unsubstituted aryl group having six to sixteen carbon atoms, a substituted or unsubstituted alkanesulfonyl group having one to ten carbon atoms, a substituted or unsubstituted arenesulfonyl group having six to sixteen carbon atoms, a substituted or unsubstituted alkoxy group having one to ten carbon atoms, a substituted or unsubstituted aryloxy group having six to sixteen carbon atoms, or a SF₅ group.

32. The process of claim 31, the arylsulfur halotetrafluoride in which X is a chlorine atom.

33. The process of claim 31, the arylsulfur halotetrafluoride in which R¹, R², R³, R⁴, and R⁵ each is independently selected from a group consisting of a hydrogen atom, a halogen atom, a substituted or unsubstituted linear or branched alkyl group having one to four carbon atoms, and a nitro group.

34. The process of claim 31, the arylsulfur halotetrafluoride in which all of R¹, R², R³, R⁴, and R⁵ are a hydrogen atom, or at maximum three of R¹, R², R³, R⁴, and R⁵ each is independently selected from a group consisting of a halogen atom, a substituted or unsubstituted linear or branched alkyl group having one to four carbon atoms, and a nitro group, and the remainders are a hydrogen atom.

35. The process of claim 31, wherein the arylsulfur halotetrafluoride is selected from a group consisting of phenylsulfur chlorotetrafluoride, o-, m-, and p-alkylphenylsulfur chlorotetrafluorides wherein the alkyl is a linear or branched alkyl group having one to four carbon atoms, o-, m-, and p-fluorophenylsulfur chlorotetrafluorides, o-, m-, and p-chlorophenylsulfur chlorotetrafluorides, o-, m-, and p-bromophenylsulfur chlorotetrafluorides, o-, m-, and p-nitrophenylsulfur chlorotetrafluorides, and each isomer of difluorophenylsulfur chlorotetrafluoride.

36. The process of claim 31, wherein the reducing substance is a substance which has reduction potential that is lower than that of arylsulfur halotetrafluoride represented by the formula (I) used in the reaction.

37. The process of claim 31, wherein the reducing substance is at least one substance selected from a group consisting of elements and inorganic and organic compounds that reduce arylsulfur halotetrafluoride represented by the formula (I) to arylsulfur trifluoride represented by the formula (II).

38. The process of claim 37, wherein the elements are alkali metals, alkali earth metals, transition metals, metals in Groups 13˜15 of the Periodic Table, and semi-metals; the inorganic compounds are inorganic chloride salts, inorganic bromide salts, and inorganic iodide salts; and the organic compounds are organic chloride salts, organic bromide salts, organic iodide salts, substituted and unsubstituted aromatic hydrocarbons, substituted and unsubstituted heteroaromatic compounds, substituted and unsubstituted unsaturated aliphatic hydrocarbons, substituted and unsubstituted nitrogen-containing aliphatic hydrocarbons, organic sulfur compounds, salts or complexes of substituted or unsubstituted heteroaromatic compounds and hydrogen fluoride, and salts or complexes of substituted or unsubstituted nitrogen-containing aliphatic hydrocarbons and hydrogen fluoride.

39. The process of claim 31, wherein the reducing substance is at least one substance selected from a group consisting of inorganic chloride salts, inorganic bromide salts, inorganic iodide salts, organic chloride salts, organic bromide salts, organic iodide salts, substituted and unsubstituted aromatic hydrocarbons, substituted and unsubstituted heteroaromatic compounds, substituted and unsubstituted unsaturated aliphatic hydrocarbons, substituted and unsubstituted nitrogen-containing aliphatic hydrocarbons, organic sulfur compounds, salts or complexes of substituted or unsubstituted heteroaromatic compounds and hydrogen fluoride, and salts or complexes of substituted or unsubstituted nitrogen-containing aliphatic hydrocarbons and hydrogen fluoride.

40. The process of claim 31, wherein the reducing substance is at least one arylsulfur compound having a formula (IIIa) or a formula (IIIb) as follows; 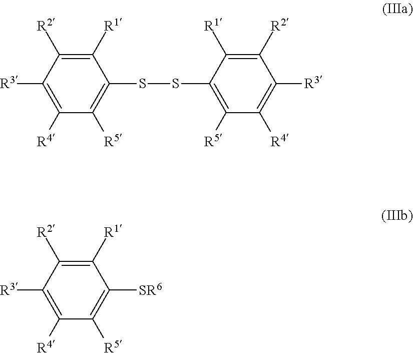
wherein R^{1′}, R^{2′}, R^{3′}, R^{4′}, and R^{5′} each is independently a hydrogen atom, a halogen atom, a substituted or unsubstituted linear, branched, or cyclic alkyl group having one to ten carbon atoms, a nitro group, a cyano group, a substituted or unsubstituted aryl group having six to sixteen carbon atoms, a substituted or unsubstituted alkanesulfonyl group having one to ten carbon atoms, a substituted or unsubstituted arenesulfonyl group having six to sixteen carbon atoms, a substituted or unsubstituted alkoxy group having one to ten carbon atoms, a substituted or unsubstituted aryloxy group having six to sixteen carbon atoms, or a SF₅ group, and R⁶ is a hydrogen atom, a halogen atom, a metal atom, an ammonium moiety, a phosphonium moiety, or a silyl moiety.

41. The process of claim 31, wherein the reducing substance is LiCl, NaCl, KCl, RbCl, CsCl or mixture thereof.

42. The process of claim 31, wherein the reducing substance is at least one substance selected from a group consisting of pyridine and its derivatives.

43. The process of claim 31, wherein the reducing substance is at least one substance selected from a group of alkyl alkenyl ethers.

44. A process of preparation of arylsulfur trifluoride represented by the formula (II); 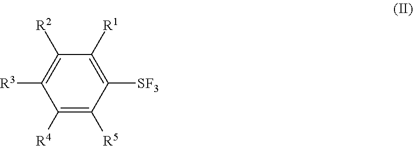
comprising contacting arylsulfur halotetrafluoride represented by the formula (I) with a reducing substance. 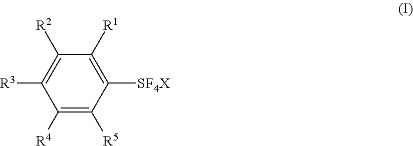
wherein X is a chlorine, bromine, or iodine atom, and R¹, R², R³, R⁴, and R⁵ each is independently a hydrogen atom, a halogen atom, a substituted or unsubstituted linear, branched, or cyclic alkyl group having one to ten carbon atoms, a nitro group, a cyano group, a substituted or unsubstituted aryl group having six to sixteen carbon atoms, a substituted or unsubstituted alkanesulfonyl group having one to ten carbon atoms, a substituted or unsubstituted arenesulfonyl group having six to sixteen carbon atoms, a substituted or unsubstituted alkoxy group having one to ten carbon atoms, a substituted or unsubstituted aryloxy group having six to sixteen carbon atoms, or a SF₅ group.

45. A process of claim 44, wherein the reducing substance is a substance which has reduction potential that is lower than that of arylsulfur halotetrafluoride represented by the formula (I) used in the reaction.

46. A process of claim 44, wherein the reducing substance is at least one substance selected from a group consisting of elements and inorganic and organic compounds that reduce arylsulfur halotetrafluoride represented by the formula (I) used in the reaction.

47. The process of claim 46, wherein the elements are alkali metals, alkali earth metals, transition metals, metals in Groups 13˜15 of the Periodic Table, and semi-metals; the inorganic compounds are inorganic chloride salts, inorganic bromide salts, and inorganic iodide salts; and the organic compounds are organic chloride salts, organic bromide salts, organic iodide salts, substituted and unsubstituted aromatic hydrocarbons, substituted and unsubstituted heteroaromatic compounds, substituted and unsubstituted unsaturated aliphatic hydrocarbons, substituted and unsubstituted nitrogen-containing aliphatic hydrocarbons, organic sulfur compounds, salts or complexes of substituted or unsubstituted heteroaromatic compounds and hydrogen fluoride, and salts or complexes of substituted or unsubstituted nitrogen-containing hydrocarbons and hydrogen fluoride.

48. The process of claim 44, wherein the reducing substance is at least one substance selected from a group consisting of inorganic chloride salts, inorganic bromide salts, inorganic iodide salts, organic chloride salts, organic bromide salts, organic iodide salts, substituted and unsubstituted aromatic hydrocarbons, substituted and unsubstituted heteroaromatic compounds, substituted and unsubstituted unsaturated aliphatic hydrocarbons, substituted and unsubstituted nitrogen-containing aliphatic hydrocarbons, organic sulfur compounds, salts or complexes of substituted or unsubstituted heteroaromatic compounds and hydrogen fluoride, and salts or complexes of substituted or unsubstituted nitrogen-containing aliphatic hydrocarbons and hydrogen fluoride.

49. A process of claim 44, wherein the reducing substance is at least one arylsulfur compound having a formula (IIIa) or a formula (IIIb) as follows; 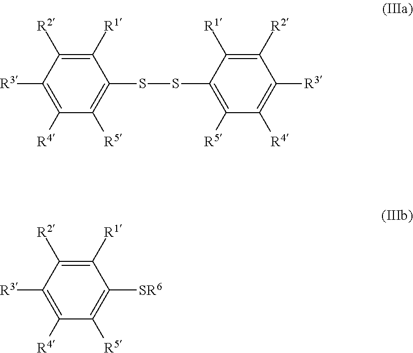
wherein R^{1′}, R^{2′}, R^{3′}, R^{4′}, and R^{5′} each is independently a hydrogen atom, a halogen atom, a substituted or unsubstituted linear, branched, or cyclic alkyl group having one to ten carbon atoms, a nitro group, a cyano group, a substituted or unsubstituted aryl group having six to sixteen carbon atoms, a substituted or unsubstituted alkanesulfonyl group having one to ten carbon atoms, a substituted or unsubstituted arenesulfonyl group having six to sixteen carbon atoms, a substituted or unsubstituted alkoxy group having one to ten carbon atoms, a substituted or unsubstituted aryloxy group having six to sixteen carbon atoms, or a SF₅ group, and R⁶ is a hydrogen atom, a halogen atom, a metal atom, an ammonium moiety, a phosphonium moiety, or a silyl moiety.

50. A process of claim 44, wherein the reducing substance is LiCl, NaCl, KCl, RbCl, CsCl, or mixture thereof.

51. A process of claim 44, wherein the reducing substance is at least one substance selected from a group consisting of pyridine and its derivatives.

52. A process of claim 44, wherein the reducing substance is at least one substance selected from a group consisting of alkyl alkenyl ethers.
